(19) United States
(12) Patent Application Publication
(10) Pub. No.: US 2024/0382410 A1
(43) Pub. Date: Nov. 21, 2024

(54) ROBOTIC MICROCAPSULE ASSEMBLIES WITH EMERGENT MOBILITY FOR TARGETED TREATMENT AND DRUG DELIVERY

(71) Applicant: THE TRUSTEES OF THE UNIVERISTY OF PENNSYLBANIA, Philadelphia, PA (US)

(72) Inventors: Hyun Koo, Philadelphia, PA (US); Edward Steager, Media, PA (US); Daeyeon Lee, Wynnewood, PA (US); Hong-Huy Tran, Philadelphia, PA (US)

(73) Assignee: THE TRUSTEES OF THE UNIVERISTY OF PENNSYLBANIA, Philadelphia, PA (US)

(21) Appl. No.: 18/668,785

(22) Filed: May 20, 2024

Related U.S. Application Data
(60) Provisional application No. 63/503,386, filed on May 19, 2023.

(51) Int. Cl. A61K 9/00; A61K 9/50; A61K 47/69

(52) U.S. Cl. CPC A61K 9/0009; A61K 9/501; A61K 47/6925

(57) ABSTRACT

The disclosed subject matter provides systems and methods for delivering a therapeutic agent for targeted treatments. The system can include at least one microcapsule and a magnetic system. The microcapsule can include the therapeutic agent and a layer of iron oxide nanoparticles (IONPs). The therapeutic agent can be encapsulated by the layer of the IONPs that include a first type of nanoparticles and a second type of nanoparticles. The magnetic system can be configured to generate a magnetic field to control the motion of the microcapsule as well as to assemble into multi-capsule structure and control its motion. Assemblies of multiple microcapsules can be controlled simultaneously and directed to targeted areas for delivery of larger therapeutic payload.

### CROSS-REFERENCE TO RELATED APPLICATION

This application claims priority to U.S. Provisional Application Ser. No. 63/503,386 filed May 19, 2023, the contents of which are hereby incorporated by reference in its entirety.

### STATEMENT REGARDING FEDERALLY-SPONSORED RESEARCH

This invention was made with government support under DE025848 and DE029985 awarded by the National Institutes of Health. The government has certain rights in the invention.

### BACKGROUND

Certain techniques have been developed for targeted medical treatments. For example, certain targeted drug delivery systems like liposomes, micelles, macromolecular conjugates, framework nucleic acids, and biodegradable nanoparticles can improve therapeutic outcomes by directing specific ligand-receptor binding or enhancing permeability and retention effects. However, the application of these passive delivery systems can be limited without active propulsion mechanisms because their accumulation and distribution are primarily governed by fluid circulation and diffusion due to concentration gradients, often failing to reach the target of interest.

Certain microbotics have the potential for improving targeted treatments. For example, such microrobots could be guided to targeted areas for localized and controlled drug release, minimizing side effects and maximizing effectiveness. Unfortunately, challenges regarding directed mobility and navigation persist. Furthermore, surface coating or grafting can be a common cargo-loading strategy for microrobots, but this can result in limited payload volume and poor drug protection during transport or limited release in the targeted area. Mixing therapeutic agents with precursors before building robots, such as 3D printing resins or hydrogels, can also cause complications like drug denaturation or leakage, and limited loading and release on-site. Drug protection can be a broader challenge influenced by factors beyond loading strategies. Such challenges in directed motion, loading capacity, cargo protection, and on-demand delivery can lead to poor drug protection, denaturation, or leakage, as well as ineffective delivery to the infection site.

Accordingly, there is an unmet need for improved techniques and systems for improved targeted treatment and drug delivery.

### SUMMARY

The disclosed subject matter provides techniques for delivering a therapeutic agent on-site with high precision and high drug protection and payload. An example system includes at least one microcapsule and a magnetic system. The at least one microcapsule can include the therapeutic agent and a layer of iron oxide nanoparticles (IONPs). The therapeutic agent can be encapsulated by the layer of the IONPs, and the layer of the IONPs can include a first type of nanoparticles and a second type of nanoparticles. The magnetic system can be configured to generate a magnetic field to control a motion of the at least one microcapsule.

In certain embodiments, the microcapsule can be configured to release the therapeutic agent at a target region. In certain embodiments, the therapeutic agent can be a water-based therapeutic agent or an oil-based therapeutic agent. In non-limiting embodiments, the microcapsule can be configured to contain the therapeutic agent more than about 73.00% of the microcapsule volume.

In certain embodiments, the microcapsule can have an asymmetric structure. The microcapsule with the asymmetric structure can include the layer of IONPs, where the IONPs are concentrated at a portion of the layer.

In certain embodiments, at least two microcapsules form a chain-like robotic assembly by coupling the microcapsules. In non-limiting embodiments, the chain-like robotic assembly can include more than three microcapsules. In non-limiting embodiments, the robotic assembly chain can be configured to adapt to and crawl over a physical barrier to reach a target region.

In certain embodiments, a diameter of the microcapsule ranges between about 10 μm to about 1000 μm. In non-limiting embodiments, a thickness of the layer ranges from about 10 nm to about 10 μm.

In certain embodiments, the motion of the microcapsule can include rolling, walking, kayaking, spinning, or combinations thereof.

In certain embodiments, the first type of nanoparticles can be SiO₂ nanoparticles, and the second type of nanoparticles can be Fe₃O₄ nanoparticles.

In certain embodiments, the system can be configured to deliver the therapeutic agent without causing surface damage or cytotoxicity to the biological cells.

The disclosed subject matter also provides methods of delivering a therapeutic agent. An example method can include introducing at least one microcapsule to a subject, directing the at least one microcapsule to a target area using a magnetic system, and disrupting the at least one microcapsule for releasing the therapeutic agent at the target area. In non-limiting embodiments, the microcapsule can include the therapeutic agent and a layer of iron oxide nanoparticles (IONPs). The therapeutic agent can be encapsulated by the layer of the IONPs that include a first type of nanoparticles and a second type of nanoparticles. In non-limiting embodiments, the magnetic system can be configured to generate a magnetic field to control a motion of the at least one microcapsule.

In certain embodiments, the method can further include introducing the therapeutic agent before or after the microcapsule is formed.

In certain embodiments, the method can further include producing a chain-like robotic assembly by coupling at least two microcapsules. In non-limiting embodiments, the at least two microcapsules are asymmetric microcapsules.

In certain embodiments, the motion of the microcapsule and/or the chain-like robotic assembly can include a single rolling, walking, kayaking, spinning, or combinations thereof.

In certain embodiments, the directing the at least one microcapsule to the target area can include delivering the therapeutic agent to the target area without structural damage.

In certain embodiments, a structure of the microcapsule can be disturbed at a target region by a physical force, a thermal energy, or a magnetic force.

In certain embodiments, the directing the at least one microcapsule to the target area can include directing the at least one microcapsule to overcome physical barriers, fluid shear, adhesive tissues, confined spaces, sticky surfaces, or combinations thereof by inducing the disclosed motions.

In certain embodiments, the method can further include accumulating at least two microcapsules to the target area to increase a drug payload release.

The disclosed subject matter will be further described below, with reference to example embodiments shown in the drawings.

### BRIEF DESCRIPTION OF THE DRAWINGS

FIG. 1 provides images showing the formation of robotic microcapsules using droplet-templated microfluidics fabrication in accordance with the disclosed subject matter.

FIG. 2 provides an image showing the formation of double emulsions from three fluid flows inside a microfluidic device in accordance with the disclosed subject matter.

FIG. 3 provides images showing the 3-axis Helmholtz coil system and schematic illustrations of the robotic motions of microcapsules in accordance with the disclosed subject matter.

FIG. 4 provides images showing the fabrication of asymmetric microcapsules in accordance with the disclosed subject matter.

FIG. 5 provides images showing the directed assembly of microcapsules to generate desirable configurations under a precessing magnetic field in accordance with the disclosed subject matter.

FIG. 6 provides images showing that the assembly of asymmetric robotic microcapsules overcomes a staircase-like obstacle under an out-of-plane rotating magnetic field in accordance with the disclosed subject matter.

FIG. 7 provides images showing the assemblies of asymmetric robotic microcapsules walking over the surface of human gingival cells without causing damage to the cells under an out-of-plane rotating magnetic field in accordance with the disclosed subject matter.

FIG. 8 provides images showing that the assemblies of asymmetric robotic microcapsules overcomes a tissue surface with complex topography and adhesive surface under an out-of-plane rotating magnetic field in accordance with the disclosed subject matter.

FIG. 9 provides images showing in vitro biocompatibility with human gingival cells with the assembly of asymmetric robotic microcapsules walking over using an out-of-plane rotating magnetic field in accordance with the disclosed subject matter.

FIGS. 10A-10F provide diagrams showing an example hierarchically assembled reconfigurable microrobots in accordance with the disclosed subject matter.

FIGS. 11A-11H provide diagrams and graphs showing an example fabrication, dynamics, and assembly behavior of magnetic microcapsules in accordance with the disclosed subject matter.

FIGS. 12A-12H provide diagrams and graphs showing an example dynamic stability of robotic assemblies in accordance with the disclosed subject matter.

FIGS. 13A-13D provide diagrams showing example adaptability and reconfigurability of an asymmetric magnetic microcapsule assembly for overcoming a stair-like obstacle in accordance with the disclosed subject matter.

FIGS. 14A-14D provide diagrams showing adaptability and reconfigurability for potential targeted biomedical applications over palatal tissue in accordance with the disclosed subject matter.

FIG. 15 provides a cryo-SEM image showing an example microcapsule after oil evaporation in accordance with the disclosed subject matter.

FIG. 16 provides a cross-sectional SEM image showing an example microcapsule shell in accordance with the disclosed subject matter.

FIG. 17 provides a graph showing an example magnetic susceptibility of individual microcapsules measured using superconducting quantum interference device (SQUID) measurement in accordance with the disclosed subject matter.

FIG. 18 provides a diagram showing a 3D FEMLAB simulation of the magnetic field distribution and energy density of an asymmetric magnetic microcapsule chain with a linear chain-like configuration in accordance with the disclosed subject matter.

FIG. 19 provides a photo showing the collected palatal tissue in accordance with the disclosed subject matter.

FIG. 20 provides a photo showing an asymmetric magnetic microcapsule assembly that adopts a zig-zag configuration on the surface of a palatal tissue in accordance with the disclosed subject matter.

FIGS. 21A-21D provide a schematic illustration of magnetically responsive catalytic microcapsules for efficient treatment of root canal infections in accordance with the disclosed subject matter.

FIGS. 22A-22G provide diagrams showing example preparation, characterization, and catalytic activity of catalytic microcapsules in accordance with the disclosed subject matter.

FIG. 23 provides photos and diagrams showing example navigation in the confined space mimicking the root canal and catalysis of microcapsules in accordance with the disclosed subject matter.

FIGS. 24A-24F provide photos and graphs showing example application scenarios of robotic microcapsules in accordance with the disclosed subject matter.

FIG. 25 provides images showing an example controlled release mechanism of microcapsules through mechanical forces to induce payload release in accordance with the disclosed subject matter.

FIGS. 26A-26F provides diagrams and graphs showing catalytic activity of the disclosed magnetic microcapsules in accordance with the disclosed subject matter.

It is to be understood that both the foregoing general description and the following detailed description are exemplary and are intended to provide further explanation of the disclosed subject matter.

### DETAILED DESCRIPTION

The disclosed subject matter provides systems and methods for targeted treatments and drug delivery. The disclosed subject matter provides a platform for targeted treatments and on-site drug delivery based on robotic microcapsule assemblies by merging characteristics from microfluidics fabrication, directed assembly, magnetic technique, and robotic principles.

Unless otherwise defined, all technical and scientific terms used herein have the same meaning as commonly understood by one of ordinary skill in the art. In case of conflict, the present document, including definitions, will control. Certain methods and materials are described below, although methods and materials similar or equivalent to those described herein can be used in the practice or testing of the presently disclosed subject matter. All publications, patent applications, patents, and other references mentioned herein are incorporated by reference in their entirety. The materials, methods, and examples disclosed herein are illustrative only and not intended to be limiting.

As used in the specification and the appended claims, the singular forms “a,” “an,” and “the” include plural referents unless the context clearly dictates otherwise. Thus, for example, a reference to “a compound” includes mixtures of compounds.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value.

An “individual” or “subject” herein is a vertebrate, such as a human or non-human animal, for example, a mammal. Mammals include, but are not limited to, humans, primates, farm animals, sport animals, rodents, and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

The disclosed subject matter provides a robotic microcapsule assembly with emergent mobility. In certain embodiments, the disclosed robotic entity can work as single or multiple units in concert to load, carry and deliver a predetermined dose of diverse drug payloads with exceptional accuracy, protection, and emergent mobility in a fully automated process.

In certain embodiments, the robotic microcapsule can include iron oxide nanoparticles (IONPs) and a therapeutic agent. In non-limiting embodiments, the therapeutic agent can be encapsulated by a layer of IONPs. For example, a therapeutic agent can be encapsulated by a layer that includes SiO₂ nanoparticles and Fe₃O₄ nanoparticles.

In certain embodiments, the diameter of the microcapsule can range from about 10 μm to about 1000 μm. In non-limiting embodiments, the size of the microcapsule can be adjusted depending on the volume of the therapeutic agents, the geometry of the target area, and the subject. In non-limiting embodiments, the therapeutic agent, which can be encapsulated inside a microcapsule, can include chlorhexidine, stannous fluoride, hydrogen peroxide, ketoconazole, clotrimazole, triiodothyronine, β-glycerophosphate, biologics (e.g., antimicrobial peptides, lipids, lectins, mRNA/siRNA, antibodies, enzymes, etc.), small molecules (e.g., flavonoids, terpenoids, polyphenols, sesquiterpenes, etc.), and living cells (e.g., bacteria). In non-limiting embodiments, the disclosed IONPs with a size of less than 100 nm and a hydrophobic surface can be suitable for the fabrication of robotic microcapsules described herein.

In certain embodiments, the thickness of the nanoparticle layer can range from about 10 nm to about 10 μm. The microcapsule can be configured to contain the therapeutic agent more than about 73.00% of the microcapsule volume (e.g., about 5.25×10⁻⁵ to about 5.25×10⁻¹ μL or diameter from about 10 μm to about 1000 μm).

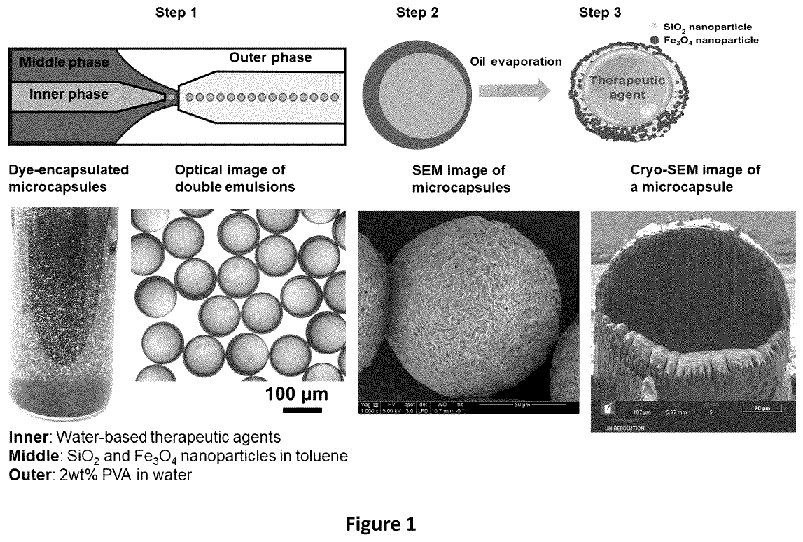

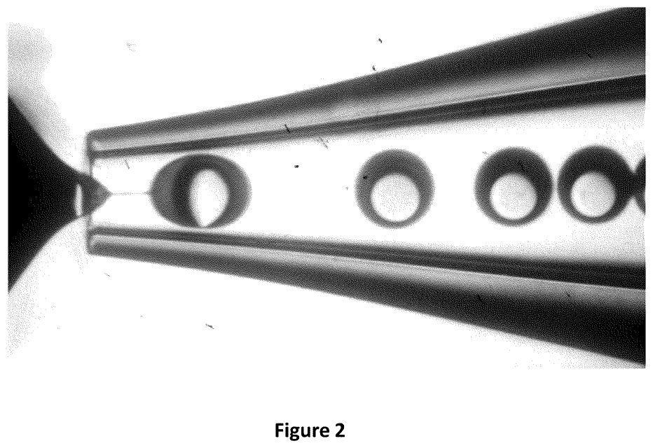

In certain embodiments, the robotic microcapsule assembly can be created through microfluidic fabrication techniques. For example, the robotic microcapsules are created through a three-part process: 1) hydrophobic iron oxide nanoparticles and silica nanoparticles are dispersed within an oil phase; 2) this mixture is then used to create water-in-oil-in-water (W/O/W) double emulsions through droplet microfluidics; and 3) the oil is subsequently evaporated to generate microcapsules, each consisting of a liquid core encased within a shell of nanoparticles surrounded by a semi-rigid, protective nanoparticle shell. In non-limiting embodiments, water can be used as the inner and outer phases and a volatile organic solvent (e.g., toluene) as the middle phase. In non-limiting embodiments, for stable emulsions, suspension of IONPs can be mixed with suspension of hydrophobic SiO₂ NPs and toluene at a predetermined volume ratio (e.g., 1 (SiO₂ NP suspension): 4 (IONP suspension): 1 (toluene)). An example concentration of nanoparticles in the oil phase can be about 40 wt % for SiO₂ NPs and about 8 wt % for IONPs. In non-limiting embodiments, the size of the double emulsions and the resulting microcapsules can be precisely tuned by controlling the flow rates of each fluid phase. The schematic illustration for fabricating robotic microcapsules using microfluidics and their morphology at each state are shown in FIG. 1. In non-limiting embodiments, a microfluidic device with flow focusing and co-flowing geometry can be used to generate double emulsions (see FIG. 2 showing the formation of double emulsions from three fluid flows inside a microfluidic device).

In certain embodiments, the robotic microcapsules and microcapsule assemblies can be controlled by magnetic fields. The disclosed magnetic fields can be used to manipulate and direct the behaviors of robotic systems. The magnetic fields can be up-and-coming for medical use because of their minimally invasive deep tissue penetration and well-established clinical safety. For example, a 3-axis Helmholtz coil system can be used to control robotic microcapsules as a single unit (or entity) or multiple units to form assemblies with varying units and collective behaviors (e.g., inter-unit interactions, mobility and locomotion). The magnetic system can include three pairs of orthogonal coils that can produce uniform magnetic fields in the X, Y, and Z axes, allowing precise manipulation of the robots' movements and orientation within the controlled environment. The motion of the robotic microcapsules can also be controlled by other magnetic systems, including electromagnets in coil or saddle configurations, permanent magnets or combinations of electromagnets and permanent magnets. In non-limiting embodiments, the robotic microcapsules and their assemblies can be manipulated by a computer-based control system, which can receive input commands from a predefined algorithm. The system then can translate these commands into corresponding current values for each coil pair, enabling precise control of the robots within the generated magnetic field. In non-limiting embodiments, the robotic microcapsules can be operated using a computer-based control system. This control system receives input commands from a predefined algorithm. For instance, the algorithm can instruct the robotic microcapsules to move in a specific direction or perform a task that requires a particular type of motion. The control system then translates these commands into appropriate current values for each coil, allowing for accurate control of the robotic microcapsules within the magnetic field generated by the system.

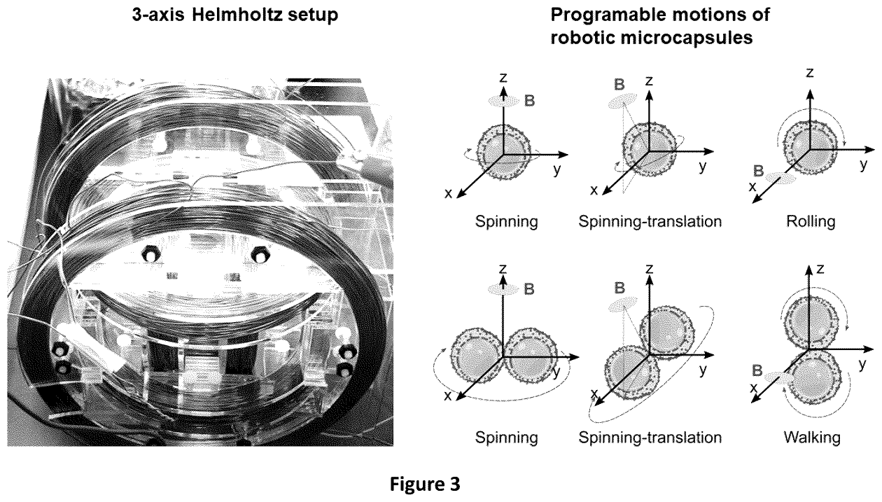

In certain embodiments, the disclosed robotic microcapsules can be reversibly assembled and disassembled on demand by controlling the external magnetic field. For example, once assembled, precise control over the magnetic field allows engineering the single unit or collective locomotion/mobility of robotic microcapsules through tuned pairwise magnetic dipole-dipole interactions to form microcapsule assemblies. Several motions can be achieved, including single rolling (out-of-plane rotation), walking (out-of-plane rotation of more than two microcapsules), kayaking (inclination motion), and spinning (in-plane rotation). Schematic illustrations of the motions are shown in FIG. 3.

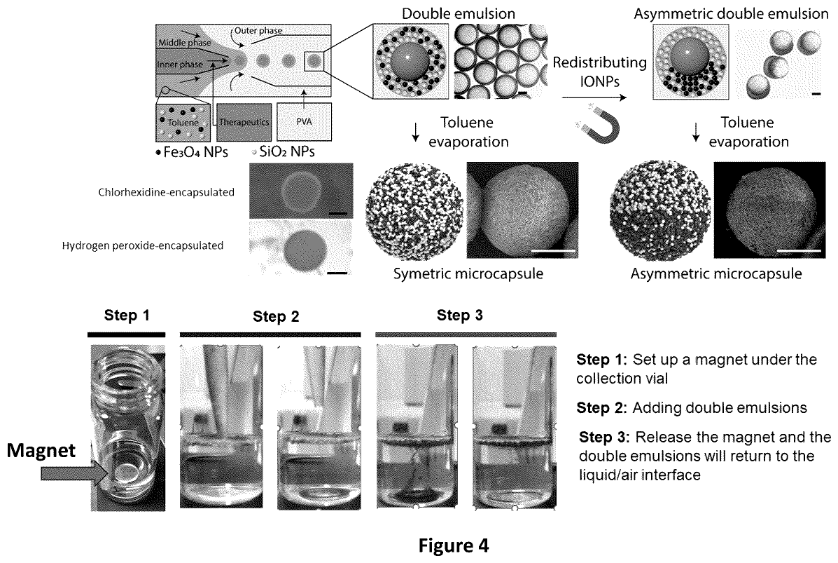

In certain embodiments, the disclosed robotic system can include a single microcapsule or multiple microcapsules. In non-limiting embodiments, more than three robotic microcapsules can work together, forming the robotic system using the microcapsules with asymmetric structure. The microcapsule with the asymmetric structure can have nanoparticles concentrated at a portion of the nanoparticle layer. For example, the upper limit number of microcapsules per robot for this type of non-improved robotic microcapsule can be 3. An unstable configuration, which shows assembly and disassembly, can be observed for assemblies with more than 3 microcapsules per robot. To enhance the magnetic interaction between microcapsules, consequently leading to the assembly of even higher-ordered structures, a predetermined level of the magnetic field can be applied at the double emulsions during the manufacturing process (e.g., at 2 in FIG. 1). By applying a magnetic field, the magnetic nanoparticles can be redistributed to be concentrated on one side, consequently forming asymmetric microcapsules (FIG. 4). In certain embodiments, using these asymmetric microcapsules, a stable multi-microcapsule chain can be formed during their movements. For example, more than 4 microcapsules (e.g., 7 microcapsules) can be formed into a 700 μm chain and move stably.

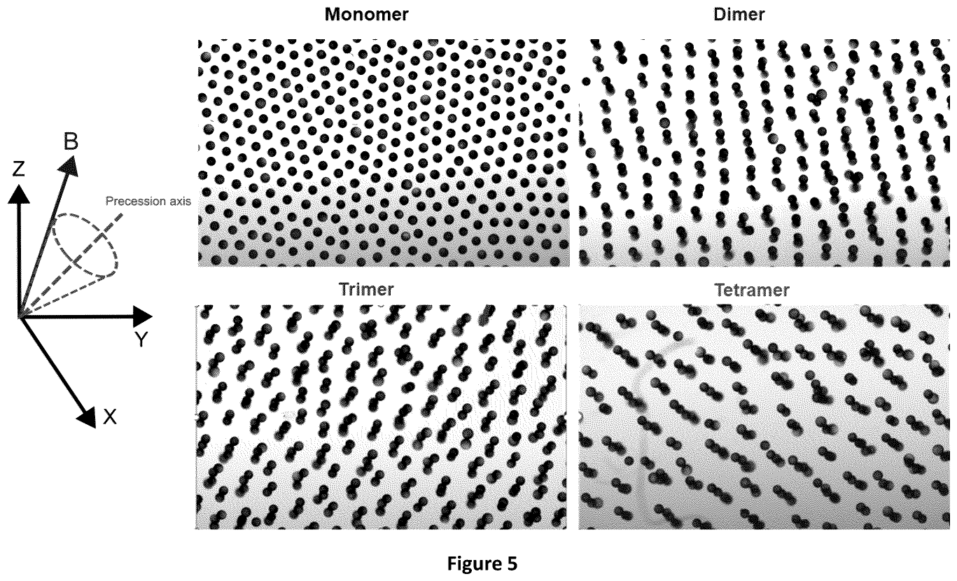

In non-limiting embodiments, the disclosed system can direct the assembly of microcapsules to generate desirable configurations and change motions automatically, with the potential for performing tasks on demand (FIG. 5). The robotic microcapsules can be precisely controlled using a precessing magnetic field. This enables the robotic microcapsules to form specific configurations (such as monomer, dimer, trimer, and tetramer) with a high distribution ratio across a wide field of view. This feature, coupled with the microcapsules' ability to form dynamically stable assemblies, can provide a formidable resistance against fluid streams and the ability to overcome physical obstacles. Due to this formidable resistance against fluid streams/ability to overcome obstacles, the disclosed system can be used for various applications within the biomedical field or any environment where fluid flows and uneven surfaces are prevalent. In non-limiting embodiments, by harnessing attractive or repulsive magnetic forces, commands with improved precision can be generated for the distribution of the disclosed robotic microcapsules, clustering them densely and in high numbers in a localized region to concentrate the drug molecules or spreading them across a larger area.

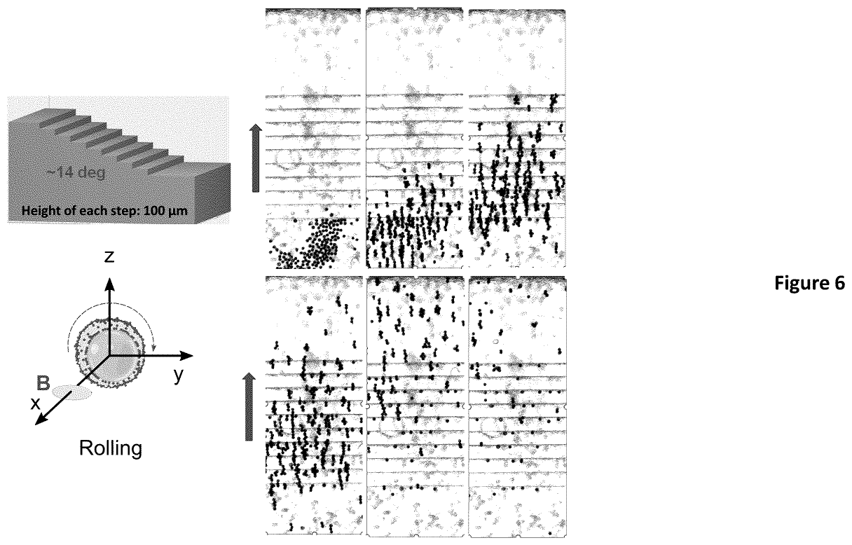

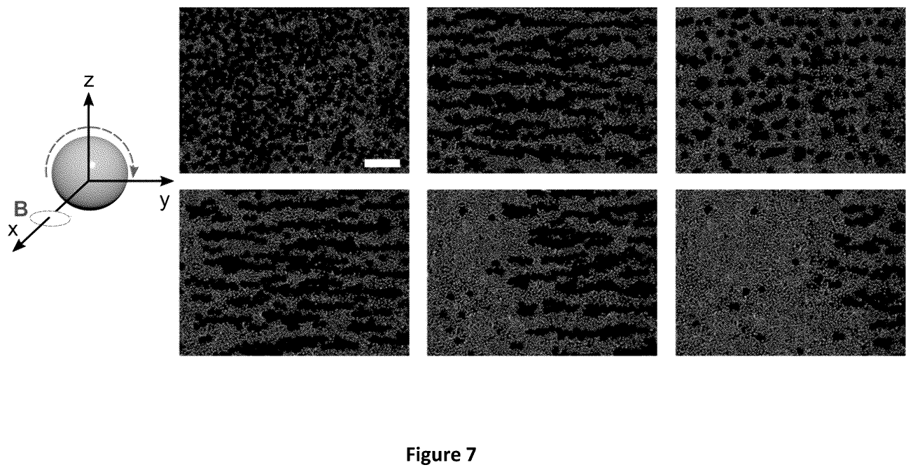

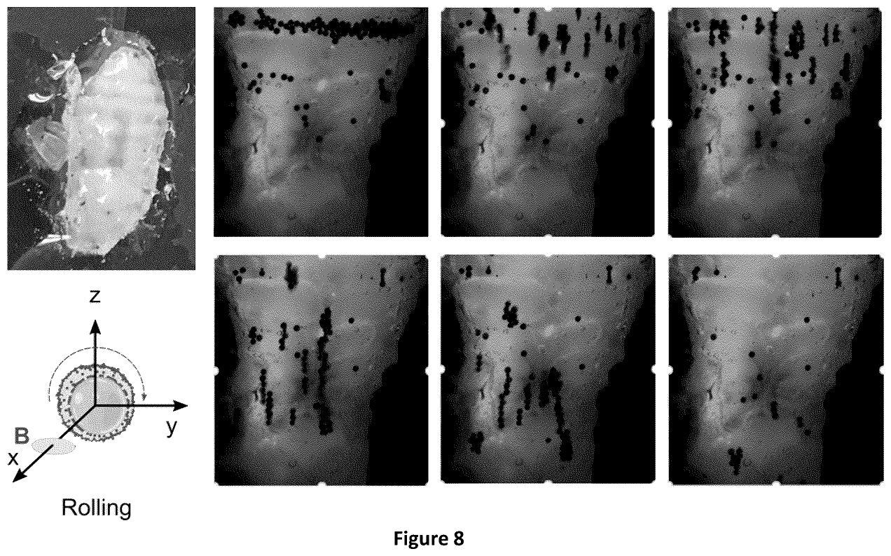

In certain embodiments, the disclosed system can generate the collective behavior of the microcapsules. For example, the disclosed system can generate the collective behavior and motion of the asymmetric microcapsule robots for controlled propulsion as magnetically responsive platforms by enhancing their translocation across physical barriers (FIG. 6, the height of each step: 100 μm) and sticky cell (FIG. 7) and tissue surfaces (FIG. 8) without physically harming or damaging. For example, using the magnetic field system, a precise command can be generated to make the assemblies of the microcapsule chain crawl over the various barriers and surfaces without damage by rolling the microcapsules with asymmetric structures.

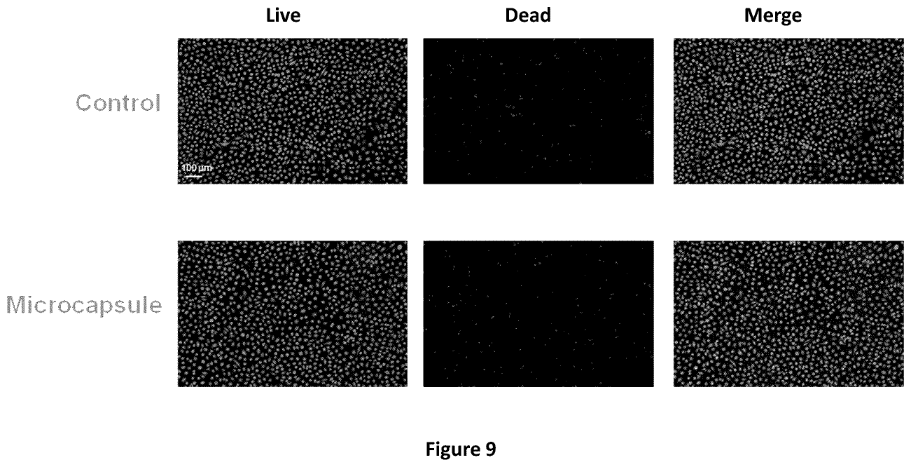

In certain embodiments, the disclosed system can deliver the therapeutic effect without damage to a target or a subject. For example, the disclosed robotic microcapsules do not exhibit any toxicity or cause physical damage to cells in vitro (FIG. 9). Thus, it can be advantageous for the disclosed robotic microcapsule assemblies to be applicable in many real-world applications without hindering the motility and function of one another. Examples of potential active agents that can be encapsulated inside a microcapsule include chlorhexidine, stannous fluoride, hydrogen peroxide, ketoconazole, clotrimazole, triiodothyronine, β-glycerophosphate, biologics (antimicrobial peptides, lipids, lectins, mRNA/siRNA, antibodies, enzymes, etc.), small molecules (flavonoids, terpenoids, polyphenols, sesquiterpenes, etc.), and living matters (e.g, bacteria).

In certain embodiments, the disclosed microcapsules can protect the loaded therapeutic agent from outside environments and deliver it to the target area. In non-limiting embodiments, the microcapsule can include a high volume of the therapeutic agent (e.g., more than about 73.00% of the microcapsule volume) and overcome the physical barriers, fluid shear, and sticky tissues and surfaces to reach the target area. For example, using the disclosed magnetic system, the microcapsule can move as a swarm (e.g., collectives) and be directed to a target area, overcoming the physical barriers, fluid shear, and sticky tissues and surfaces through various motions. In non-limiting embodiments, more than one microcapsule can be accumulated on the target area for increased drug payload release.

The encapsulation can provide high drug payload incorporation and payload protection. The magnetic system can be configured to generate a magnetic field to control the motion of one microcapsule or a collection of multiple microcapsules. Microcapsules can display emergent properties under the magnetic field that allow co-assemblies into multi-unit structures that can be moved individually, or several units can be moved collectively. This collective behavior can allow unprecedented mobility to overcome physical hurdles, sticky surfaces and shear fluid while directing and clustering at a targeted location for high drug concentration delivery on-site with precision.

The disclosed subject matter also provides methods of targeted treatments or delivery of therapeutic agents. The method can include introducing at least one microcapsule to a subject. The introduced microcapsule can include a therapeutic agent and a layer of IONPs, where the therapeutic agent can be encapsulated by the layer of the IONPs. In non-limiting embodiments, the layer of the IONPs comprises different types of nanoparticles.

In certain embodiments, the method can further include directing the microcapsule to the target area using a magnetic system. The magnetic system can be configured to generate a magnetic field to control the motion of the microcapsule. For example, the magnetic system can generate the magnetic field to make the microcapsule rolling (e.g., out-of-plane rotation), walking (e.g., out-of-plane rotation of more than two microcapsules), kayaking (e.g., inclination motion), or spinning (e.g., in-plane rotation). In non-limiting embodiments, the magnetic system can be configured to control the motion of the microcapsules in a fully automated process. For example, microcapsules can follow a predetermined algorithm, exhibiting specific motions at specified time points. For instance, they can be magnetically configured to form a group, walk for a set time, spin, change direction, and repeat to follow a predetermined path. In non-limiting embodiments, the microcapsule can deliver the therapeutic agent to the target area without any damage to surrounding environments and tissues.

In certain embodiments, the method can further include disrupting the microcapsule to actively release the therapeutic agent at the target area. For example, when the microcapsule reaches the target area, the structure of the microcapsule can be disturbed by physical force, thermal energy, ultrasound force, or magnetic force. In non-limiting embodiments, the therapeutic agent can be released through diffusion, controlled by a nanoparticle shell enveloping the cargo. This process can be calibrated, with the release profile depending on the molecular weight of the encapsulated agents. Alternatively, mechanical forces can be used to break open the shell and release the payload. The mechanically induced stimulus can be safe and effective for therapeutic release in medical applications. Furthermore, by incorporating IONPs into the shell, on-demand rupture of the microcapsules can be achieved using thermal stimulation. For example, thermal stimulation can be achieved by inducing rapid magnetic field oscillation, allowing precise control.

In certain embodiments, the method can further include producing a robotic assembly chain by coupling at least two microcapsules. For example, the chain-like robotic assembly can be produced by coupling at least two microcapsules with the disclosed asymmetric structure. By coupling the microcapsules with the disclosed asymmetric structure, more than three microcapsules can be coupled to form the robotic chain.

In certain embodiments, the method can further include introducing the therapeutic agent into the microcapsule. In non-limiting embodiments, the therapeutic agent can be introduced before or after the microcapsule is formed. For example, the microcapsule can be formed first with a cavity, and the therapeutic agent can be infused into the cavity. For example, the microcapsule can be formed first with a plain water core, and the therapeutic agent can be infused by swapping out this water core, utilizing a concentration gradient. In another example, the therapeutic agent can be encapsulated when the layer of IONPs is formed for the microcapsules.

The disclosed subject matter provides microrobots with collective behavior. The disclosed microbots can provide solutions for the issues of limited mobility, restricted payload capacity, drug protection, and effective delivery to the targeted site, especially helpful at the microscale. The disclosed subject matter provides various reliable methods for delivering and releasing the encapsulated cargo upon robotic microcapsules reaching their designated location/target.

With the nanoscale size, the building blocks can be dissolved and safely eliminated post-release. Alternatively, taking advantage of the magnetic properties of the robotic microcapsules, the remaining particles and microcapsules can be magnetically retrieved once the drug is successfully delivered at the target site. The disclosed subject matter, therefore, can address the challenges of the field and introduce reliable techniques for payload delivery and recovery of the carriers.

The disclosed subject matter provides solutions to various issues related to targeted treatment by integrating the principles of microfluidics fabrication, directed assembly, and robotics. The disclosed robotic entity can carry and deliver a predetermined dose of diverse drug payloads on-site through their emerging mobility and collective behaviors in a fully automated process. In non-limiting embodiments, the disclosed system can include asymmetric microcapsule assemblies. These structures can boast an internal chamber for abundant drug storage and spatial separation, all securely encased within a sturdy and protective nanoparticle shell. This design allows high drug loading, assures drug protection and mobility/locomotion across different physical and surface constraints/barriers, and enables controlled release in a localized manner at the targeted location/site. Under an external magnetic field, they uniquely demonstrate collective behaviors, varied translational movements, switchable motion modes, and pinpoint navigation. The disclosed subject matter provides the superior locomotion capability of microrobots and the unique liquid-encapsulating characteristics of microcapsules, illustrating a versatile avenue for developing an improved platform for targeted treatments.

In certain embodiments, the disclosed subject matter provides a microrobotic platform that can provide hierarchical assembly and multi-directional control of microcapsule assemblies for targeted drug delivery and catalysis activation within the root canal. The disclosed system can navigate complex, confined root canal morphologies, delivering multiple drugs simultaneously and performing localized catalysis or accelerated reactive oxygen species (ROS) exposure on demand. In non-limiting embodiments, the disclosed system can be configured to carry different drug mixtures, and the motion behaviors can be controlled.

The disclosed systems and techniques involve microrobots crafted from magnetic microcapsules with improved drug-loading capabilities, designed for improved mobility and reconfigurability in complex biological environments. The disclosed microrobots, assembled using droplet microfluidics and magnetic control, can exhibit collective mobility and self-reconfiguration, enabling them to navigate through adhesive and rugged tissues as well as confined/narrow spaces, thereby enhancing targeted delivery of high therapeutic payloads in biomedical applications.

### Example 1: Hierarchically Assembled Reconfigurable Microrobots for Traversing Rugged and Adhesive Surfaces

Microrobots can be poised to transform biomedicine and clinical applications through non-invasive methodologies and precision targeting. However, they can face challenges in adapting to and navigating complex and adhesive biological environments. In this example, using droplet microfluidics and magnetic control, iron oxide nanoparticles (10 nm) are assembled hierarchically into 100-micron-size asymmetric microcapsules to form submillimeter, multi-unit robotic assemblies. The asymmetry is a critical feature that facilitates the formation of robust assemblies under rotating magnetic fields. Assemblies of these microcapsules exhibit unexpected collective mobility with higher degrees of freedom to create cohesive yet self-reconfigurable structures. These structures mimic kinematic chains, consisting of a sequence of spherical joints, allowing for multi-axis rotational motions. The modular nature of microcapsule assemblies enables them to rotate, bend at different angles, and self-reconfiguration to traverse complex physical obstacles significantly larger than their size and traverse adhesive biological tissues that typically restrict the movement of microscale systems.

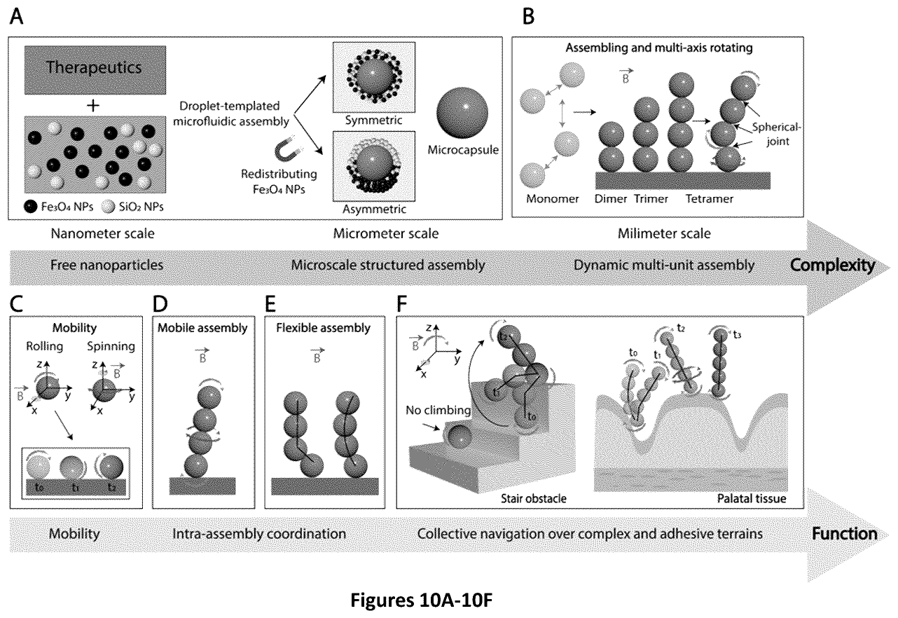

The disclosed subject matter introduces a hierarchical assembly approach, spanning from nanometers to hundreds of micrometers, to create a robotic platform composed of asymmetric magnetic microcapsules (FIG. 10) with at least the following features: (1) localized magnetic energy interaction between microcapsules (assembly units) creates a highly stable robotic chain; (2) the multi-unit assembly provides stable yet bendable structure with rotational motion dynamics resembling ‘spherical joint multi-degree-of-freedom’; (3) the robotic assemblies provide emergent properties where they can self-reconfiguration their structure under the rotating magnetic field providing a dynamic mechanism of adaptability and reconfigurability to overcome physically and biologically challenging topographies.

The fabrication of magnetic microcapsules involves, with reference to FIG. 10A,: (1) forming water-in-oil-in-water double emulsions using droplet-templated microfluidics, with iron oxide nanoparticles and silica nanoparticles dispersed in the oil phase; (2) self-assembling nanoparticles through droplet templating in the oil phase, encapsulating the water phase as the core; and (3) evaporating the oil to obtain microcapsules with a liquid core surrounded by a nanoparticle shell. For asymmetric magnetic microcapsule fabrication, a magnetic field is applied during (2) when the magnetic nanoparticles are still freely dispersed in the oil phase to induce fluid-magnetic trapping, localizing these magnetic nanoparticles to one side of the oil phase of double emulsions and later in the shell of microcapsules.

The dynamics and assembly behaviors of these magnetic microcapsules are studied under a uniform magnetic field generated from a three-axis Helmholtz coil system. This uniform magnetic field applies a magnetic torque on the microcapsule to align it with the magnetic field vectors. Local interactions between microcapsules through magnetic dipole-dipole interactions make them assemble into robotic chain-like structures, as shown in FIG. 10B. The magnetic field can be programmed to induce the microcapsules to exhibit different motion modes (e.g., rolling and spinning) in any plane of 3D space, as shown in FIG. 10C and 10D.

Leveraging fluid-magnetic trapping to localize the magnetic nanoparticles in one region of the shell, local magnetic interactions between microcapsules, which provide unique self-reconfigurablity to form robust yet flexible microrobot assemblies, can be engineered (FIG. 10E). The disclosed fabrication strategy provides the ability to create symmetrical or asymmetrical distributions of magnetic nanoparticles in the shell of microcapsules. Because of the robustness and modularity, the assemblies of asymmetric magnetic microcapsule can bend at different angles, rotate at intersections, and adapt their structures around an obstacle to overcome topographically challenging physical (stair-like obstacle) and adhesive biological (mice palatal tissue) barriers (FIG. 10F). FIG. 10 shows the hierarchical assembly of asymmetric magnetic capsules for self-reconfigurable modular microrobots: (10A) Droplet-templated microfluidics fabrication of magnetic microcapsules; (10B) Dynamic assembly of magnetic microcapsules under the magnetic fields that self-adjust into stable, bendable, and rotational robotic chains resembling multi-degree-of-freedom spherical joints; (10C) Types of motion under magnetic maneuverability of magnetic microcapsules; (10D) Synchronized motion of magnetic microcapsules upon forming assemblies; (10E) Reconfiguration of magnetic microcapsule assemblies; (10F) Magnetic microcapsule assemblies can self-reconfigure and adapt to overcome physical and biological obstacles.

Symmetric and asymmetric magnetic microcapsules: Magnetic microcapsules are fabricated using the water-in-oil-in-water double emulsions-templated microfluidics method, with iron oxide nanoparticles (IONPs) dispersed in the oil phase (toluene) to incorporate magnetic properties. To generate stable double emulsions, a suspension of 40 wt. % hydrophobic silica (SiO₂) nanoparticles in toluene is mixed with the 8 wt. % IONPs-in-toluene suspension. Stable emulsions require an optimal volume ratio (1 (SiO₂ NP suspension): 4 (IONP suspension): 1 (toluene)). These hydrophobic SiO₂ nanoparticles stabilize the double emulsions by adsorbing to the inner/middle and middle/outer interfaces. A 2 wt. % solution of polyvinyl alcohol (PVA) in water is used as both the aqueous inner and outer phases. This PVA solution improves the stability of double emulsions by adsorbing to the oil-water interfaces, thereby preventing their coalescence.

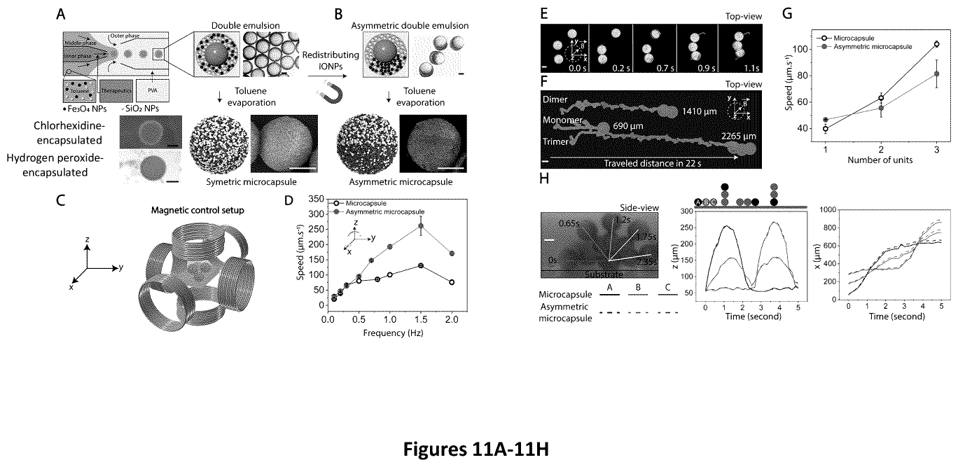

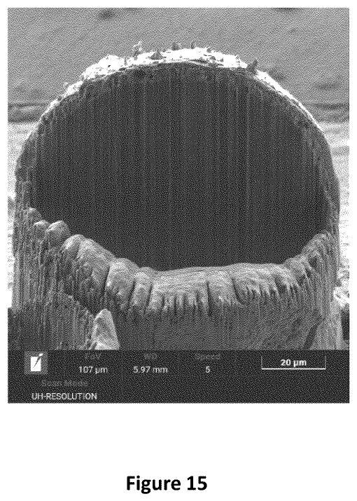

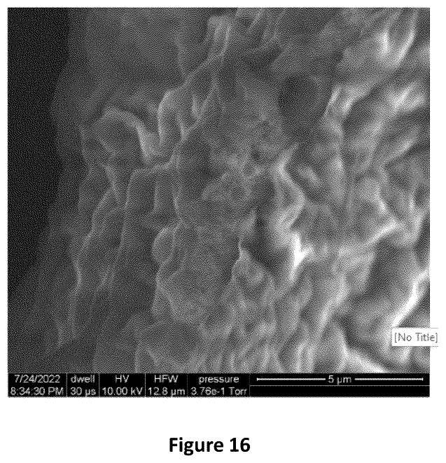

The double emulsions present a uniform core-shell morphology with inner and outer diameters of ˜100 μm and ˜110 μm, respectively (FIG. 11A). Interestingly, the distribution of magnetic nanoparticles can be manipulated. For example, an applied magnetic field (˜30 mT) for 30 s induces the formation of double emulsions with an eccentric oil shell by localizing IONPs in one side of the oil shell (FIG. 11B). Given the high concentration of SiO₂ nanoparticles and IONPs and the rapid evaporation of toluene, the magnetic fluid (IONPs suspension) remains trapped in one side of the oil shell by magnetically unresponsive SiO₂ nanoparticles even after the applied magnetic field is removed. The ability to localize and trap the magnetic nanoparticles provides an effective strategy for tuning capsule-capsule interactions under magnetic fields. The symmetric magnetic microcapsules and their asymmetric counterparts are obtained upon evaporating the oil phase. IONPs and SiO₂ nanoparticles, initially dispersed in the oil phase, become the shells of microcapsules upon removal of toluene. The localization of magnetic nanoparticles prior to the solvent removal does not induce any noticeable change in the overall morphology of the microcapsules. These microcapsules have an outer diameter of ˜100 μm, exhibiting a core-shell structure wherein a liquid cargo is encapsulated by a nanoparticle shell with an average thickness of ˜1.65 μm (FIG. 11A and B and FIGS. 15-16).

Dynamics of individual magnetic microcapsules: Prior to exploring the dynamic assembly behaviors, the dynamics of isolated magnetic microcapsules were analyzed, where intercapsule interactions were negligible. The mobility and dynamics of these magnetic microcapsules (with and without asymmetry of IONPs in the shell) are studied under a uniform magnetic field generated from a three-axis Helmholtz coil system. A schematic illustration of the magnetic field actuation setup is shown in FIG. 11C. Briefly, magnetic microcapsules are dispersed in the water contained in a 25 mm×25 mm×15 mm petri dish with a glass coverslip bottom, positioned at the center of the magnetic actuation system. This magnetic actuation setup produces a uniform magnetic flux density of approximately 10 mm in the x, y, and z directions. Magnetic microcapsules are highly responsive to the external magnetic fields and their motion can be controlled through magnetic torques. Multiple dynamic modes can be achieved by customizable algorithms. For instance, a rotating magnetic field in the vertical plane (x-z) can actuate the rolling motion of the magnetic microcapsules. The rotating magnetic field (B) for the rolling motion at a frequency (f) with a time-varying (t) can be expressed as follows:

$$B ( f , t ) = {B}_{m} [ cos ( 2 π ft ) u + sin ( 2 π ft ) v ] \qquad ( 1 )$$

Bₘ represents the magnitude of the magnetic field, and the unit vectors u and v represent the base vectors in the rotating planes (x and z) of the magnetic field.

As the net magnetic moment of the magnetic microcapsules aligns with the rotating magnetic field, a torque is generated when the magnetic field vector starts rotating. The magnetic torque of a rotating microcapsule generates a surface interaction force, countered by a rotational fluid drag force. Due to the physical boundaries from the surface of the substrate, the rotating microcapsule encounters an unbalanced fluid drag force. The magnetic torque then balances the fluid drag force. The unbalanced fluid drag, based on surface-induced symmetry breaking, is the primary driving force for the rolling motion of the magnetic microcapsules. By varying the magnetic actuation frequency, the rolling speed of the magnetic microcapsules can be altered (FIG. 11D). The motion of magnetic microcapsules linearly correlates with the actuation frequency of the applied magnetic field up to a critical “step-out” frequency (˜1.5 Hz) and reaches the maximum rolling speed of 130.9 μm·s⁻¹ for an individual symmetric microcapsule and 261.7 μm·s⁻¹ for an individual asymmetric microcapsule. Beyond 1.5 Hz, the relationship between the microcapsule's rolling speed and the magnetic field rotation frequency ceases to be linear because the magnetic torque cannot balance the fluid drag.

The asymmetric magnetic microcapsules show an increase in their speed when the frequency of the magnetic field is increased from 0.5 to 1.5 Hz. The enhancement of magnetic responsiveness in asymmetric magnetic microcapsules can be attributed to the asymmetric distribution of iron oxide nanoparticles. The localization of iron oxide nanoparticles not only increases the magnetic moment in specific regions of the microcapsule but also leads to an uneven distribution of magnetic forces upon exposure to a magnetic field. Consequently, this generates increased torque, facilitating more dynamic and rapid adjustments in the microcapsule's orientation and position in response to changes in the magnetic field. A magnetic field actuation frequency of 0.2 Hz was used in all subsequent analyses. This choice is made because the magnetic response of the magnetic microcapsules (with and without asymmetry of IONPs) does not show any noticeable differences at low actuation frequencies (<0.5 Hz), enabling direct comparison of their behaviors.

Dynamics of magnetic microcapsule assemblies: advantages of colloidal microrobots is the emergence of collective behaviors as they self-organize into robotic assemblies. These robotic assemblies exhibit critical functionalities, such as high mobility, adaptability, and reconfigurability, which are important for overcoming the major challenges in targeted therapies. To assess the collective motion of magnetic microcapsules, multiple microcapsules in proximity were used, and their assembly was observed under the dynamic magnetic fields.

When exposed to a rotating magnetic field in the vertical x-z plane, these magnetic microcapsules (both with and without asymmetry) roll, connect, and self-organize into robotic chains (FIG. 11E). The driving force for this dynamic assembly is the magnetic dipole-dipole interactions between microcapsules. The chains align along the direction of the applied dynamic magnetic field. The translational velocities of these assemblies, made of a different number of microcapsules (ranging from 1 to 3), were analyzed under a constant actuation frequency of 0.2 Hz. The velocity rises sharply with the number of microcapsule units (FIG. 11F and 11G). The velocity of the symmetric microcapsule assemblies is slightly faster than that of the asymmetric microcapsule assemblies. These assemblies of magnetic microcapsules move by tumbling under a rotating magnetic field in the x-z plane (FIG. 11H). Thus, this increase in velocity, corresponding to the number of assembly units, largely comes from an increased step length of a tumbling chain.

FIG. 11 shows the Fabrication, dynamics, and assembly behavior of magnetic microcapsules: Double emulsion-templated microfluidics fabrication of (A) symmetric and (B) asymmetric magnetic microcapsules; (C) Schematic illustration of a three-axis Helmholtz coil system for magnetic field actuation; (D) Translational speed of individual magnetic microcapsules under varying magnetic actuation frequencies; (E) Snapshots of an assembly process in forming a trimer assembly under the magnetic fields; (F) Motion tracking of magnetic microcapsules with different units; (G) Translational speed of magnetic microcapsules with different units; (H) Motion profile of a trimer assembly during motion through tumbling behavior. Scale bars are 50 μm.

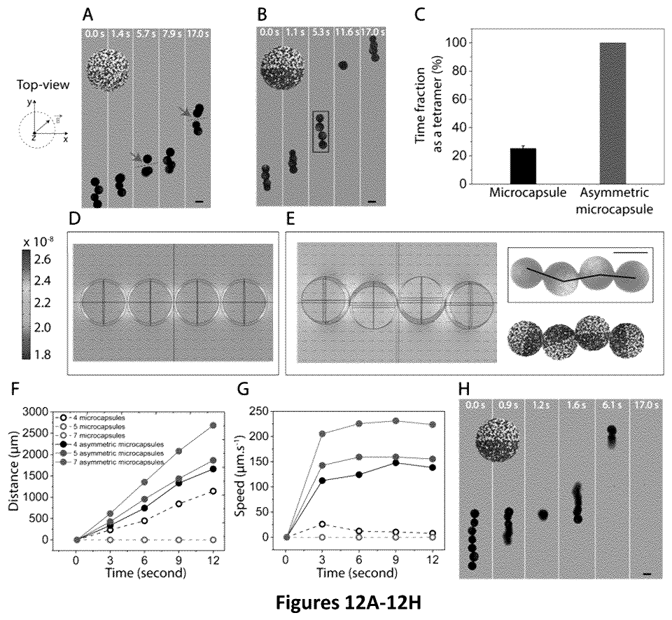

Effects of local magnetic interaction energy on dynamic stability of robotic assemblies: The magnetic microcapsules exhibit dynamic assembly and collective mobility. However, as the number of assembly units grows (i.e., longer chain), the hydrodynamic drag during rotation significantly increases, determining an upper limit for the number of microcapsules that can assemble into a single chain. The propulsion mechanism for these chain-like microcapsule assemblies mirrors that of individual units and shorter chain assemblies, relying on a solid surface for hydrodynamic symmetry breaking. Notably, the assembly of four symmetric magnetic microcapsules (tetramer) exhibits a continuous assembly-disassembly behavior during its motion, influenced by fluid drag forces (FIG. 12A). The disclosed fabrication based on droplet-templated microfluidics results in a uniform distribution of IONPs in the shell of these symmetric magnetic microcapsules. Under a rotating magnetic field, as the symmetric magnetic microcapsules attempt to form a tetramer through dipole-dipole interactions, they tend to adopt a configuration with minimal energy, allowing them to move against fluid drag forces. However, because of the uniform distribution of IONPs within the shell, when they adopt a tetramer configuration, the viscous drag forces become greater than magnetic dipole interactions. Consequently, the continuous breaking of the chain occurs when subjected to dynamic perturbations as the microcapsules strive to balance magnetic interactions and fluidic resistance, resulting in unstable assembly.

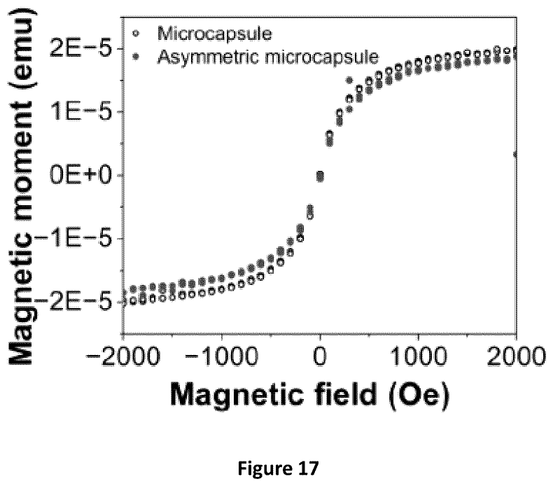

In stark contrast, the asymmetric magnetic microcapsules present a stable tetramer assembly with stronger intercapsule interactions under the same conditions (FIG. 12B). For a given period and distance, the tetramer assembly formed by symmetric microcapsules rarely maintains the tetramer chain configuration during its motion. In contrast, the tetramer assembly formed by the asymmetric microcapsules remarkably maintains its structural integrity for the entire time (FIG. 12C). Superconducting quantum interference device (SQUID) measurement indicates that the localization of magnetic nanoparticles does not induce any significant changes in the magnetic susceptibility of individual microcapsules (FIG. 17). Thus, these findings suggest that the enhanced magnetic interactions between asymmetric magnetic microcapsules do not come from a change in magnetization.

To understand the mechanisms behind the structural robustness of the microcapsule assemblies, the magnetic field distribution and energy density of chain-like configurations were modeled considering both symmetric and asymmetric microcapsule assemblies. Modeling information using 3D finite element simulations is described in detail in the Materials and Methods section. In general, for an assembly to maintain its stability, it is critical that the configuration is favorable and characterized by the maximization of magnetic energy. The magnetic energy stored in a modeled assembly is directly correlated with the magnetic field intensity in the simulation cell. The local magnetic energy density (w_{B}) for microcapsules in a vacuum follows:

$${w}_{B} = \frac{1}{2} \frac{{B}^{2}}{{μ}_{0}} \qquad ( 2 )$$

B is the magnetic field intensity, and μ₀ is the magnetic permeability of free space (4π×10⁻₇ N/A²). The total magnetic energy w_{B} of the assembly is determined by integrating w_{B} over the volume (V) of sub-domains:

$${W}_{B} = {\int }_{V} {w}_{B} d \qquad ( 3 )$$

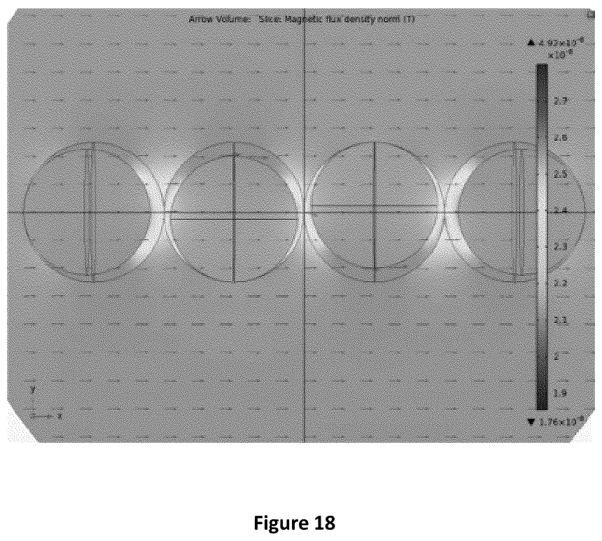

This calculation indicates that the asymmetry of the magnetic nanoparticles in the shell strengthens the structural integrity of the robotic assembly through enhanced local magnetic energy interactions. The tetramer configuration of asymmetric microcapsules has a greater magnetic energy than that of the symmetric microcapsules (FIG. 12D and 12E). Furthermore, the tetramer assemblies of asymmetric microcapsules present a slight zig-zag configuration (FIG. 12E inset). This configuration has a higher magnetic energy compared to a linear configuration (FIG. 18).

By localizing magnetic nanoparticles on one side of microcapsules, capsule-capsule forces can increase, enhancing structural integrity during motion through increased binding between microcapsules at their intersection. Regions with concentrated magnetic nanoparticles have higher magnetic dipole interaction forces. Thanks to their enhanced local interactions, more than four asymmetric microcapsules can assemble into a robotic chain, and they can tumble under the rotating magnetic field while maintaining their structural integrity, as shown in FIG. 12F. With an increase in the number of microcapsules in a single assembly, there is a notable rise in velocity, reaching a speed of approximately 225 μm·s⁻¹ when a heptamer assembly is formed (FIG. 12G). Asymmetric microcapsules can readily assemble into structures with more units (e.g., seven asymmetric microcapsules) and move without experiencing structural collapse (FIG. 12H). The enhanced local magnetic energy between neighboring asymmetric microcapsules diminishes the likelihood of spontaneous disassembly under fluid perturbations. These findings present an effective strategy for designing robotic systems where magnetic interaction energy can be engineered to enhance the dynamic interactions between subunits to create highly stable connections at their intersections that provide self-supporting assemblies with robustness for magnetic field-controlled motion.

FIG. 12 shows the dynamic stability of robotic assemblies: (12A) Snapshots of dynamic motion of a tetramer assembly made of symmetric microcapsules; (12B) Snapshots of dynamic motion of a tetramer assembly made of asymmetric microcapsules. (12C) Time fraction of tetramer assemblies during motion; (12D) and (12E) 3D simulations of the magnetic field distribution and energy density of chain-like configurations of symmetric and asymmetric systems, respectively; (12F) Traveled distance over time of magnetic microcapsule assemblies with different number of units and (12G) their corresponding velocity; (12H) Snapshots of dynamic motion of an assembly made of seven asymmetric microcapsules. Magnetic actuation frequency is 0.2 Hz. Scale bars are 100 μm.

Adaptability and reconfigurability of the asymmetric microcapsule assemblies: An important challenges in leveraging microrobots for targeted therapies is the limited mobility over surfaces with topological, chemical, and biological complexity. Here, the dynamic multi-axis interactions of asymmetric magnetic microcapsules at their intersections to form self-reconfigurable yet cohesive assemblies to navigate challenging topography through their unique mobility, adaptability, and modular reconfigurability were assessed. The navigation of asymmetric magnetic microcapsule assemblages traversing complex physical and biological barriers, such as a physical staircase and a (murine) palatal tissue, where both physical and biological (presence of rugosity on a soft and adhesive wet surface) complexities coexist, was assessed.

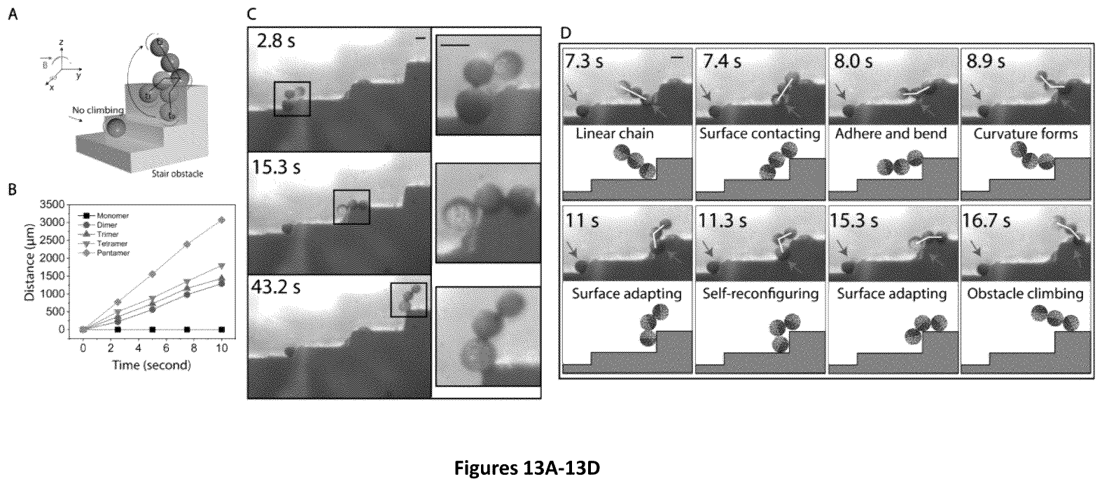

To assess the motion and navigation of asymmetric magnetic microcapsules over physical barriers, 3D-printed stair-like obstacles having multiple steps, as shown in FIG. 13A, were created. To quantify the motion of microcapsules, a stair-like obstacle with multiple uniform steps, where each step is 100 μm in height and 500 μm in width, was used. Individual asymmetric magnetic microcapsules are unable to climb up steps with heights similar to their outer diameter. In contrast, when the microcapsules form assemblies with at least two units, they can overcome this complex terrain. The results indicate that having a longer chain (more microcapsule units) is advantageous as the distance traveled over time is much greater than that of shorter chains (FIG. 13B).

To further explore the capabilities of these asymmetric magnetic microcapsule assemblages, the structure of the stair was modified by varying and increasing the height of each step. This adjustment allows us to investigate how these asymmetric magnetic microcapsule assemblages respond and adapt to a physically changing barrier. While individual asymmetric magnetic microcapsules get stuck at the first step (100 μm in height), a trimer assembly can adeptly climb over multiple steps, including steps that are above 200 μm in height (FIG. 13C). Notably, the trimer microcapsule assemblage demonstrates remarkable adaptability and self-reconfigurability through multi-axis rotation and self-reconfiguration between microcapsules in its chain-like configuration during the translocation.

Specifically, the trimer, originally adapting a linear chain-like configuration, bends significantly, forming a curved line when confronted with the physical obstacle of a high step, as shown in FIG. 13D. As the assembly climbs up the step, the top microcapsule continues to rotate by rolling on the middle microcapsule until it reaches the top surface of next step. The middle microcapsule acts as a pivot, also providing a base for the other two microcapsules to rotate and slide over while maintaining the overall integrity of the assembly. By doing so, the assembly remarkably changes its linear chain configuration to effectively adopt a right-angle configuration, enabling it to conform to the sharp edge of the stair step. The top microcapsule then becomes a new pivot that induces a strong force to detach the other two microcapsules, showing self-reconfigurable and self-sustaining properties through its unique multi-axis rotation and bendability. The microcapsule assembly then returns to a linear structure as its typical configuration. Thus, by leveraging the strong yet focused magnetic interaction energy at the capsule-capsule interface, such a bendable and self-reconfigurable hinge can take advantage of obstacle topography through the surface interaction/adhesion to adapt and traverse complex terrains. This reconfigurability is analogous to dynamic bending that can be seen in large modular self-reconfigurable robot systems (tens of centimeters scale) with dedicated electronics inside and connecting linkages between robots.

FIG. 13 shows the adaptability and reconfigurability of an asymmetric magnetic microcapsule assembly for overcoming a stair-like obstacle: (13A) A schematic illustration of a trimer assembly traversing a stair; (13B) Travelled distance over time of asymmetric magnetic microcapsule assemblies with different number of units over a stair-like obstacle having multiple steps (100 μm in height); (13C) Photos capture the stair-climbing process on steps of varied heights; (13D) A time-lapse image series presenting the adaptability and reconfigurability of the trimer assembly in overcoming challenging obstacles. Scale bars are 100 μm

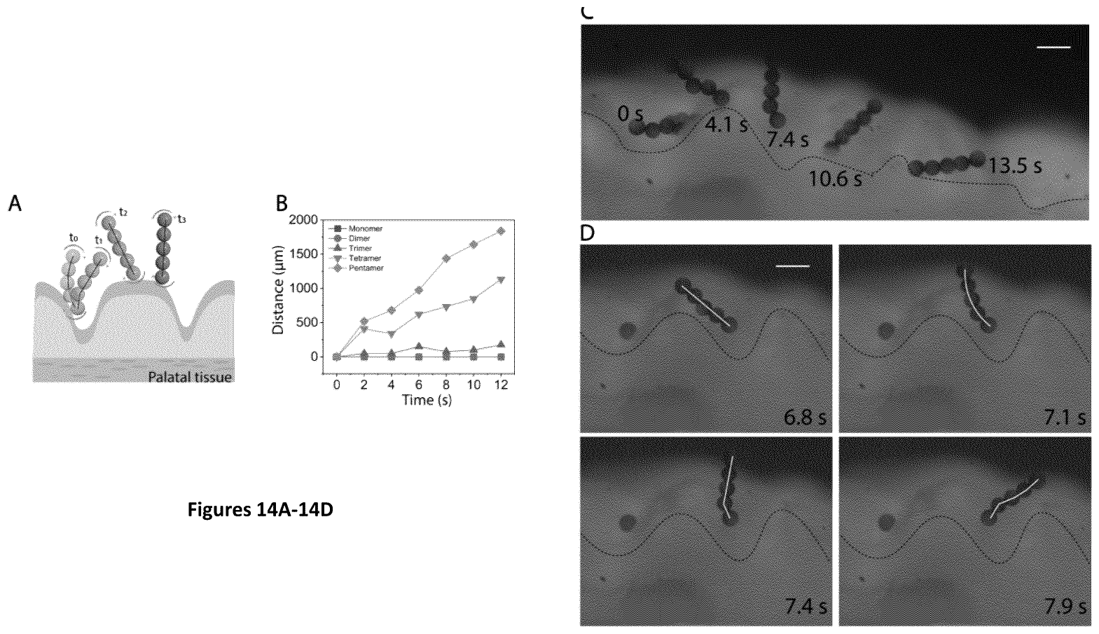

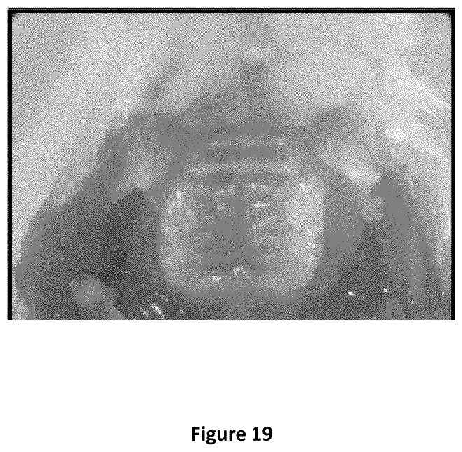

Another major hurdle for microrobot translation is navigating the complexities of unknown biological systems. Recent studies highlight a strong affinity between iron oxide nanoparticles (the primary building blocks in most microrobots) and biological cell surfaces. While increased binding enhances interactions with target cells, it can concurrently impede motion, which can cause physical entrapment and reduced mobility to reach the target of interest. To evaluate the navigation efficacy of the microcapsule assemblies, a palatal tissue model that encompasses both physical and biological complexities was used, simulating three-dimensional biological features. A schematic illustration of the experiment is shown in FIG. 14A, while the experimental image of the palatal tissue is shown in FIG. 19.

The navigation of asymmetric magnetic microcapsule assemblies on the surface of a cell layer (human immortalized gingival keratinocytes) was analyzed to assess the potential adhesion or impact of their motion on cell morphology and viability. These observations indicate that these microcapsule assemblies traverse the cellular surface without binding or causing any physical harm or damage. No significant interaction, binding, or cellular displacement due to the movement of the microcapsule assemblies is observed (FIG. 7). Additionally, the asymmetric magnetic microcapsules do not exhibit any toxicity to cells with in vitro studies (FIG. 7).

Next, the mobility and navigation of asymmetric magnetic microcapsule assemblages were assessed based on the number of units and distance traveled. The highly complex, adhesive surface of the palatal tissue significantly impedes the motion of not only individual microcapsules but also short-chain assemblies (dimer and trimer) (FIG. 14B). By analyzing the distance traveled for assemblies with different numbers of units, a minimum of four units is required for navigating the complex, adhesive surface of the palatal tissue. Thus, the pentamer assembly of asymmetric magnetic microcapsules demonstrates effective navigation across the physical and biological complexity of palatal tissue, leveraging its appropriate chain length, dynamic robustness, and self-reconfigurability (FIG. 14C).

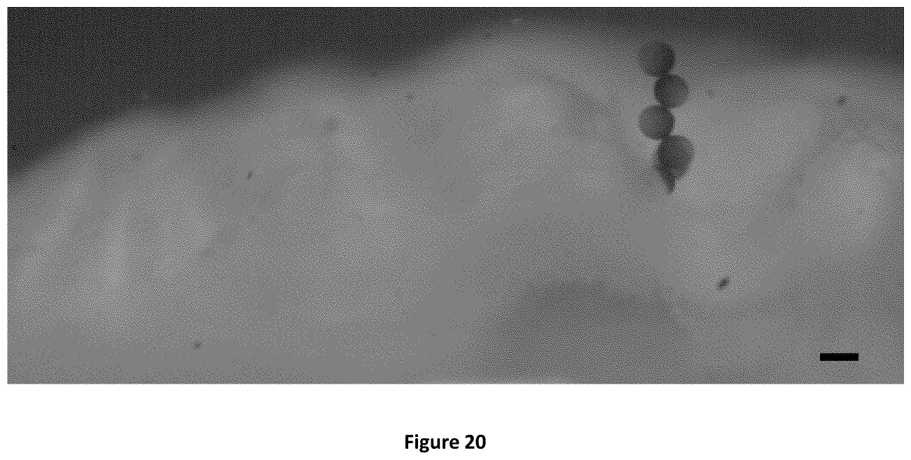

Specifically, the assembly initially adopts a typical linear chain configuration. The linear chain configuration spontaneously adjusts the arrangement of the orientation of the magnetic-rich region of the subunits, presenting a clear zig-zag structure (FIG. 20). Based on the modeling of magnetic energy interactions, this self-arrangement is to maximize capsule-capsule interactions at their intersections in the assembly. As the assembly encounters the hill-like topography of the palatal tissue, it rearranges and reconfigures its structure to exploit the asymmetry in rotation between its units to form a bendable assemblage to effectively climb over the tissue ridges (FIG. 14D).

Each intersection between asymmetric magnetic microcapsules acts as a spherical joint with roll, pitch, and twist. The microcapsule positioned at the bottom acts as a hinge, contacting and anchoring itself to the tissue surface. The anchored assembly can then bend to present a bowing configuration that could help to enhance the anchoring effect of the assembly to the tissue surface preventing sliding due to the challenging topography of the tissue. After the microcapsule at the bottom of the assembly establishes good contact with the tissue surface, the four microcapsules that are not in contact with the tissue surface leverage the spherical-joint mechanism to rotate and form a bent chain configuration. The assembly then functions as a coordinated system as the top four microcapsules self-reconfigurate and straighten the bent chain, forming an angle with the anchored microcapsule at their intersection. The top four microcapsules of the chain then keep pushing forward by narrowing the angle with the anchored microcapsule until the entire chain becomes a linear structure again while advancing. This allows the entire assembly to push forward and successfully navigate the obstacle.

Overall, this example shows that a high local magnetic interaction energy at the subunit intersections provides a unique multi-unit spherical-joint mechanism, enabling robotic assemblies to self-reconfigurate and coordinate to achieve effective motion over physical and biological barriers. This mechanism offers adaptability and self-reconfigurability, crucial for potential applications in targeted therapies and environmental exploration. FIG. 14 shows the adaptability and reconfigurability for potential targeted biomedical applications over palatal tissue: (14A) The schematic illustrates the tissue navigating process; (14B) Travelled distance over time of asymmetric magnetic microcapsule assemblies with different numbers of units over the rough tissue surface; (14C) Photos capture the tissue-climbing process; (14D) A time-lapse series presenting the adaptability and reconfigurability of the assembly in overcoming challenging biological obstacles. Scale bars is 100 μm.

In this example, a distinctive microrobotic platform was introduced, based on assemblies of asymmetric magnetic microcapsules, which demonstrates high mobility, dynamic stability, and multi-degree shape-shifting abilities through a unique spherical-joint mechanism. These robotic assemblies, created through a hierarchical assembly approach combining droplet microfluidics and magnetic control techniques, exhibit remarkable modular reconfigurability, akin to kinematic chains with multi-axis movements. These findings indicates that asymmetric distribution of magnetic nanoparticles within the microcapsule shell remarkably strengthens the local magnetic interaction energy at the assembly's intersections, enabling their dynamic adaptability without altering the magnetic susceptibility of individual microcapsules.

The fabrication process, based on double emulsion-templated microfluidics, offers versatility in tuning the structure and properties of the generated microcapsules. The disclosed magnetic-fluid trapping technique is key for redistributing and localizing magnetic nanoparticles within the microcapsule shell, affecting stability and enabling self-reconfigurability through multi-axis rotation. This ability to localize nanoparticle distribution presents an effective strategy to tailor the robotic units and optimize their functionality in response to external stimuli.

The adaptability of the magnetic microcapsule assemblies is demonstrated through their effective navigation over complex obstacles, achieved by their multi-axis self-reconfigurability. The unique spherical joint mechanism allows for multi-axis rotation at the intersections between neighboring microcapsules, enabling them to dynamically adapt to their environment. The proposed adhesion-based tumbling mechanism further illustrates how the assemblies can overcome obstacles by leveraging asymmetry in rotation among its units.

These microcapsules present opportunities to carry a wide range of payloads boosting their capability as potential active drug delivery systems. Therefore, the ability of these assemblies to target anatomically challenging and confined locations, such as the human root canal, for high-dose therapeutic delivery or in situ catalytic reactions, presents promising avenues for their application in medical interventions. Certain therapeutic-loading strategies, such as surface coating or mixing therapeutic agents with microrobot materials, often lead to poor drug protection, denaturation, or leakage. In addition, nanoparticles-based robots face challenges in increasing loading capacity and cargo protection. The presented asymmetric magnetic microcapsule assemblies can enable the ability to target anatomically challenging and confined locations. For example, magnetic microcapsule assemblies can be employed to target the apical site inside human root canal and deliver a high dose of therapeutics or induce in situ catalytic reactions at an interested sites through peroxidase-like catalytic activity from IONPs in the shell.

Overall, these findings contribute to the advancement of dynamically stable, reconfigurable microrobots and open new possibilities for their application in complex environments and targeted therapeutic delivery.

Fabrication of magnetic microcapsules using droplet-templated microfluidics: Cylindrical glass capillary tubes (1 mm outer diameter and 0.58 mm inner diameter) were pulled using a micropipette puller (P-1000, Sutter Instruments Inc.), and tapered orifices were formed with a microforge (Narishige, Japan). Orifice dimensions for inner fluid and collection were typically 50 um and 150 μm, respectively. Glass microcapillary tubes for inner fluid and collection were fitted into 1 mm inner dimension square capillary tubes, ensuring alignment for a coaxial geometry. The distance between tubes was set to ˜100 μm. Epoxy resin was used to seal these capillaries to the device platform. Solutions were delivered through polyethylene tubing via syringes controlled by syringe pumps (Harvard Apparatus, PHD 2000 series). Double emulsion formation was monitored with a high-speed camera attached to an inverted microscope.

For water-in-oil-in-water double emulsions, 2 wt. % poly (vinyl alcohol) aqueous solution (PVA, 87-89% hydrolyzed, average Mw=13000-23000, Aldrich) were used as outer and inner aqueous phases. The middle phase was prepared by mixing hydrophobic silica nanoparticle suspension (SiO₂ NPs, TOL-ST, Nissan Chemical Inc. (Japan), 15 nm diameter, 400 mg·mL⁻¹ in toluene) and iron oxide nanoparticle suspension (IONPs, EMG 1200 fatty acid coated dry nanoparticles, Ferrotec (USA), 10 nm diameter, 8 mg·mL⁻¹ in toluene). Stable emulsions require an optimal ratio (1 (SiO₂ NPs): 4 (IONPs): 1 (toluene)). The flow rate of the solutions was 1000, 1000, and 10000 μL·h⁻¹ for the inner, middle, and outer phases, respectively. For asymmetric magnetic double emulsions, a permanent magnet with approximately 30 mT of magnetic field strength was positioned beneath the double emulsion container and held for 60 seconds. Upon releasing the magnetic field, the double emulsions returned to the liquid-air interface, initiating oil evaporation. Double emulsion droplets were converted to microcapsules by evaporating the oil phase and washing with deionized water to remove the remaining oil phase.

Scanning electron microscopy (SEM) images of the microcapsules were captured using a field emission scanning electron microscope (FEI Quanta 600, FEI, Portland, OR, USA) at an acceleration voltage of 5 kV. Samples were coated with approximately 4 nm of iridium layer using a Quorom plasma-generating sputter coater prior to imaging to prevent charging.

Magnetic properties: The magnetic property analysis was conducted using Quantum Design's MPMS-XL, offering sensitive SQUID (Superconducting Quantum Interference Device) magnetometry capabilities with Quantum Design's Reciprocating Sample Option (RSO). This equipment provides a noise floor of <10-8 emu for DC moment measurements, magnetic fields up to 7 T, and temperatures typically between 2 K and 400 K. For each measurement, eight isolated microcapsules were deposited on a glass substrate (4 mm×4 mm).

Magnetic field actuation: The mobility and dynamics of these magnetic microcapsules are studied using a three-axis Helmholtz coil system (FIG. 7), generating globally uniform magnetic fields. The global magnetic field generates a torque on the magnetic microcapsules to align the microcapsule with the magnetic field vector. The external rotating magnetic field can be circularly polarized in any plane of 3D space to actuate the magnetic microcapsules in different motion modes through a customizable algorithm. To assess the dynamic behavior in a uniform magnetic field, magnetic microcapsules were dispersed in water contained in a 25 mm×25 mm×15 mm plastic petri dish with a glass coverslip bottom, positioned at the center of the magnetic actuation system. When supplied with specific voltages, this system produces uniform magnetic flux densities with a magnetic field strength of approximately 10 mm in the x, y, and z directions. By adjusting the voltage across specific coil pairs, distinct magnetic flux orientations and resultant motion behaviors are controlled.

Motion observation and analysis: The motion and assembly formation of microcapsules under the magnetic field were observed using a Zeiss Axio Zoom. V16 fluorescence upright stereo zoom microscope system (Carl Zeiss Microscopy GmbH, Jena, Germany) equipped with a 1× objective at a video frame rate of up to 100 fps for top-view imaging. A high-speed camera (CHRONOS 1.4, Kron Technologies Inc., Canada) was used to capture these behaviors from the side with the assistance of an LED shining from the opposite direction of the camera. The camera continuously recorded the movement, and post-experiment, the images were extracted for precise calculations of object movement speed based on the distance covered in a specific time frame. ImageJ software was employed to process the videos, determining the position, velocity, and dynamic behaviors of microcapsule assemblies. Particle tracking was done through the “Trackmate” function in ImageJ, where collected images were inverted before processing.

Magnetic interaction energy simulations: Three-dimensional (3D) magnetostatic calculations were performed using the Multiphysics modeling package (COMSOL, Burlington, MA) to obtain the magnetic field distribution and magnetic energy distribution around the microcapsules. The geometry of the microcapsule assemblies was represented in a 2D cross-sectional top view at the midpoint of the experimental image (FIG. 12A and 12B). Microcapsule assemblies were horizontally arranged in simulations, with the magnetic field direction from left to right. The solution space was divided into two sub-domains: water media and a thin iron oxide shell symmetrically or asymmetrically surrounding the inner core.

The physical properties values, including magnetic permeability (μ) and magnetic susceptibility (χà) for each sub-domain material. Four microcapsules arranged symmetrically or asymmetrically in a tetramer assembly configuration were placed inside the simulation box. A homogenous magnetic field of 10 mT was applied from left to right in the simulation, aligning with the longer axis of the configured assembly. The simulation was initiated, and the solution space was triangulated into a conformal mesh, which was subsequently refined. The program solved the Maxwell equations for all elements to obtain magnetic field intensity and magnetic energy density within the simulation cell. The magnetic energy of the entire tetramer configuration was calculated using the subdomain integration function. The calculations were iterated with progressively refined mesh sizes until reaching a sufficiently small mesh for the final calculated values.

Preparation of 3D-printed objects: Supporting objects, such as the sample holder in the magnetic actuation setup and the stair-like obstacles, were designed using Onshape computer-aided design software (Onshape Inc., Cambridge, USA). The designed objects were subsequently 3D-printed using a low-force stereolithography 3D printer (Form 3B, Formlabs Inc., MA, USA) with a layer thickness resolution of 50 μm. Biocompatible photopolymer resin (Dental SG V1 resin, Formlabs Inc., MA, USA) was utilized for the printing process. Following the print, the 3D-printed parts were immersed in 99% isopropanol for 20 minutes and air-dried for 30 minutes. Subsequently, the dried objects underwent UV curing (405 nm light at 60°° C.) for 60 minutes using a FormCure device (Formlabs Inc., MA, USA).

Palatal tissue preparation: a whole-organ explant culture of murine oral mucosa was performed. This procedure was approved and conducted in accordance with the University of Pennsylvania's Institutional Animal Care and Use Committee (IACUC) protocol (IACUC #806682). Briefly, the oral palatal tissues (4 mm×2 mm) were extracted from C57BL/6 mice and then cultured in MEM α media (15% FBS, 2 mM L-glutamine, 100 μM ascorbic acid, 100 U·mL⁻¹ penicillin, and 100 μg·mL⁻¹ streptomycin) at 37°° C. with 5% CO₂ for 24 hours. For imaging and analysis, tissues were secured onto glass coverslips using biological glue (PeriAcryl® 90, Glustitch). Subsequently, the tissue-containing substrate was introduced into the testing chamber filled with water as the medium. Microcapsules were gently deposited onto the tissue surface using micropipettes.

In vitro cytotoxicity: Immortalized human gingival keratinocytes (HGKs) were cultured to form a confluent monolayer. HGK cells were seeded in KBM-2 medium (Lonza Group AG, Basel, Switzerland) supplemented with 0.15 mM CaCl₂ (5,000 cells/cm²) and were incubated in a 5% CO₂ humidified atmosphere. After 24 hours, allowing the formation of the confluent monolayer, the medium was removed, and the Petri dishes were gently rinsed with Dulbecco's phosphate-buffered saline (pH=7.2). 50 μL of asymmetric magnetic microcapsules were introduced in serum-free KBM-2 medium and controlled to perform walking motion for 10 minutes. Cell viability was assessed using a LIVE/DEAD Viability/Cytotoxicity Kit for mammalian cells (L3224, Invitrogen). Briefly, serum-free KBM-2 containing 0.002 mmol calcein-AM and 0.002 mmol ethidium homodimer-1 was added to each sample. Imaging of the cells was performed using Zeiss Axio Zoom. V16 fluorescence upright stereo zoom microscope system. The experimental procedure was replicated in three independent experiments, each conducted in triplicate.

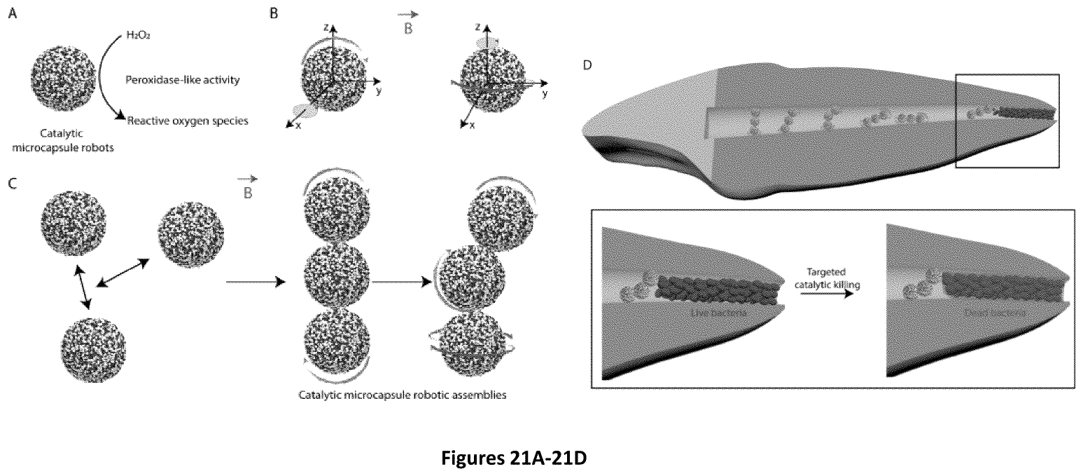

FIGS. 21A-21D show the operation of the magnetically responsive catalytic microcapsules for the treatment of root canal infections. FIGS. 21A-21D shows an illustration in using catalytic robotic microcapsules for targeting confined biofilm infections in root canal.

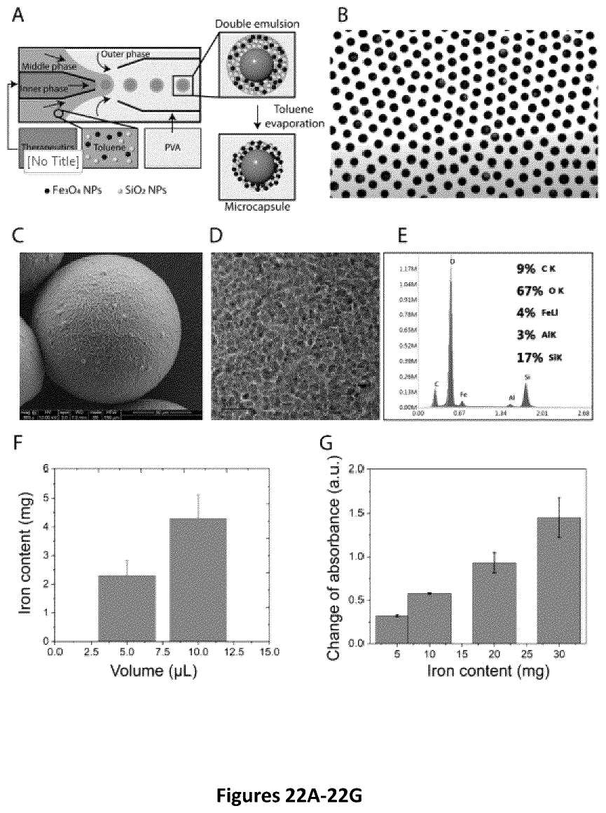

FIGS. 22A-22G show the preparation, characterization, and catalytic activity of catalytic microcapsules. FIG. 22A shows an example fabrication method of catalytic microcapsules through a droplet-templated microfluidic technique. FIG. 22A provides a schematic illustration of fabrication of magnetic microcapsules using droplet-templated microfluidic approach. FIG. 22B shows an optical image of as-prepared microcapsules. FIG. 22C shows SEM images of as-prepared microcapsules, and FIG. 22D shows TEM images of IONPs used in forming the shell. FIG. 22E shows an energy-dispersive X-ray spectroscopy (EDS) spectrum illustrating the elemental presence within a microcapsule. FIG. 22F shows the iron content in a volume of microcapsule solution through ICP analysis. FIG. 22G shows the catalytic activity of microcapsules at 1% _{H2O2} after 5 minutes of incubation using 3,3′,5,5′-tetramethylbenzidine (TMB) as a probe. The production of hydroxyl radicals can be confirmed by the photoluminescence method using coumarin as a hydroxyl radical trapping agent.

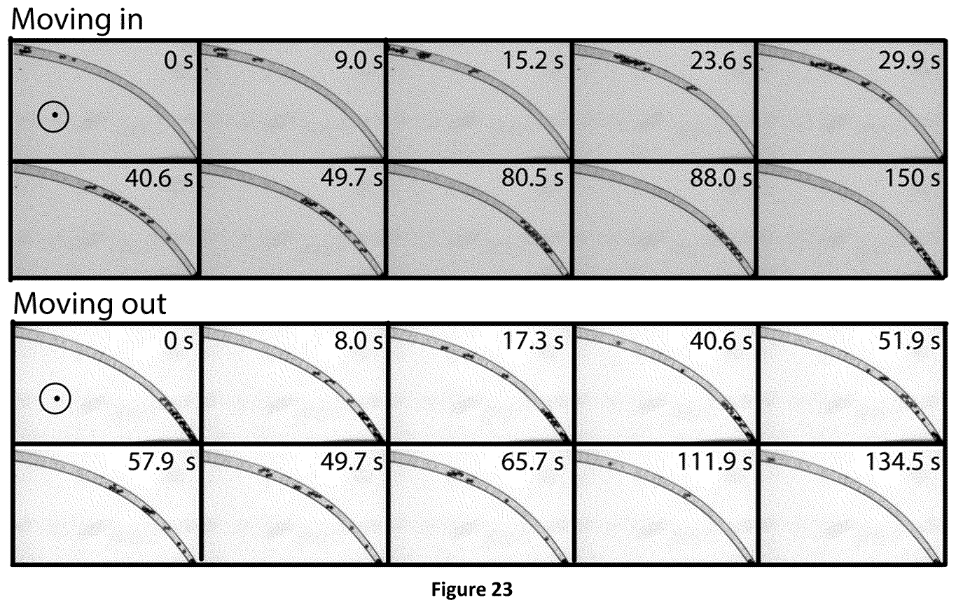

FIG. 23 shows the navigation of microcapsules (IN-and-OUT) inside a 3D-printed root canal (transparent block).

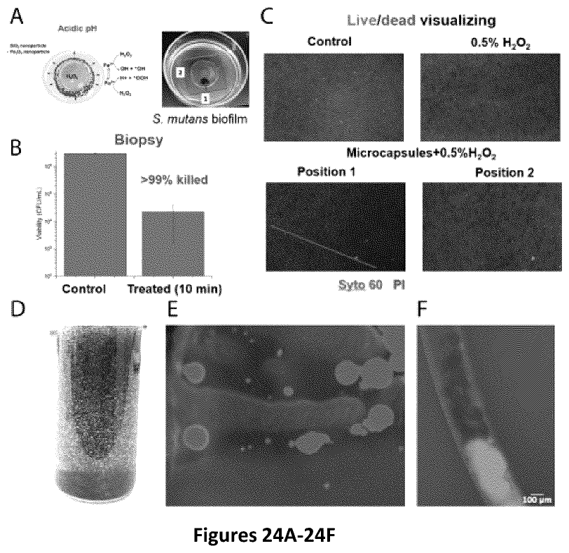

FIGS. 245A-24F shows various applications of the disclosed robotic microcapsules. FIG. 24A shows the localized catalytic killing of microcapsules. FIG. 24B shows the quantitative killing efficiency of S. mutans biofilms after 5 min catalytic treatment with microcapsules using CFU analysis. FIG. 24C shows the Live/Dead imaging of the localized killing of microcapsules under catalytic conditions. FIG. 24D shows the chlorhexidine-encapsulated microcapsules (dyed with RhB) from microfluidic fabrication. FIG. 24E whos a fluorescent image of CHX-encapsulated microcapsules (dyed with RhB) on the palatal tissue (shown as green). FIG. 24F shows the targetability of microcapsules (black particles) in treating biofilm infections (shown as green) at the apical site of the root canal.

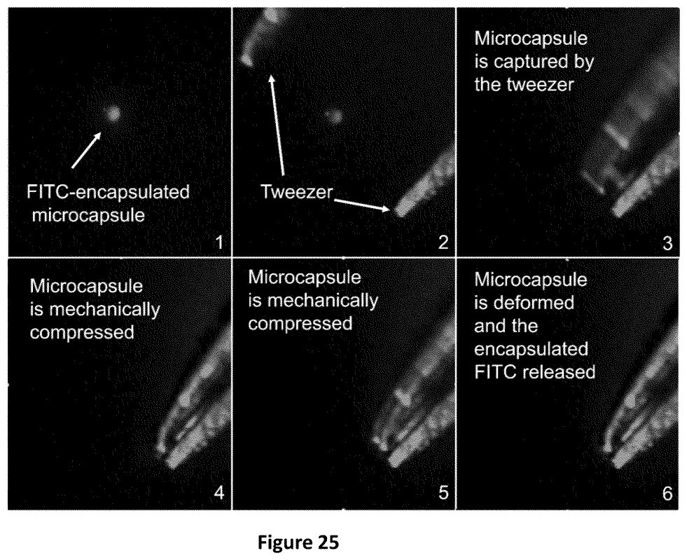

FIG. 25 shows the controlled release mechanism of the disclosed microcapsules through mechanical forces to induce the payload release. The disclosed microcapsules can include the structure of a hard nanoparticle shell, which can protect the encapsulated payload. As shown in FIG. 28, the payload, FITC, remains intact within the microcapsule, showing no signs of leakage or degradation under normal conditions. Leakage can occur when a predetermined level of mechanical force is applied, such as by using a tweezer to puncture the capsule. This controlled release mechanism highlights the potential for leveraging mechanical forces to induce payload release in various applications.

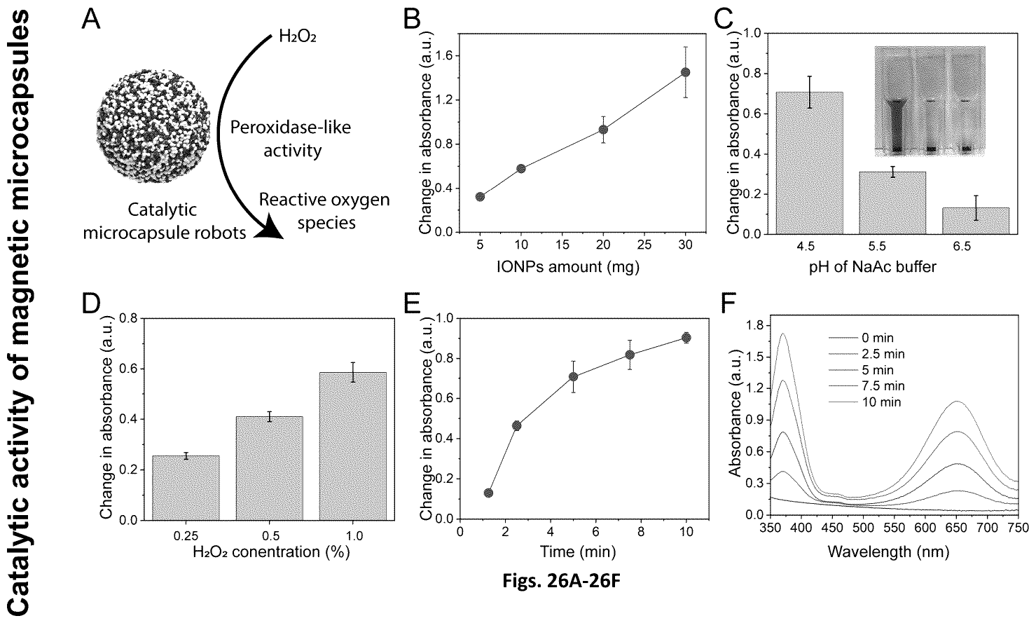

FIGS. 26A-26F show catalytic activities of the robotic microcapsules through peoroxidase-like activity using the 3,3′,5,5′-tetramethylbenzidine (TMB) colorimetric assay. FIG. 26A shows the catalytic mechanism through peoroxidase-like activity. FIG. 26B shows the change of catalytic activity in dependence of the amount of microcapsules. FIG. 26C shows the peroxidase-like activity of microcapsules at three pH values (4.5, 5.5, and 6.5) as determined by the colorimetric TMB assay. FIG. 26D shows the peroxidase-like activity of microcapsules in dependence of the concentration of H₂O₂. FIGS. 26E and 26F show the kinetic of the peroxidase-like activity of microcapsules over time as determined by the colorimetric TMB assay.

All patents, patent applications, publications, product descriptions, and protocols cited in this specification are hereby incorporated by reference in their entirety. In case of a conflict in terminology, the present disclosure controls.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain, using no more than routine experimentation, many equivalents to the specific embodiments described herein. Such equivalents are intended to be encompassed by the following claims.

What is claimed is:

1. A system for delivering a therapeutic agent comprising:
at least one microcapsule, including the therapeutic agent and a layer of iron oxide nanoparticles (IONPs), wherein the therapeutic agent is encapsulated by the layer of the IONPs, wherein the layer of the IONPs comprises a first type of nanoparticles and a second type of nanoparticles; and
a magnetic system configured to generate a magnetic field to control a motion of the at least one microcapsule.

2. The system of claim 1, wherein the microcapsule is configured to release the therapeutic agent at a target region.

3. The system of claim 1, wherein the microcapsule has an asymmetric structure, wherein the microcapsule with the asymmetric structure comprises the layer of IONPS, where the IONPs are concentrated at a portion of the layer.

4. The system of claim 1, wherein at least two microcapsules form a robotic assembly chain by coupling the microcapsules.

5. The system of claim 4, wherein the robotic assembly chain comprises more than three microcapsules.

6. The system of claim 4, wherein the robotic assembly chain is configured to crawl over a physical barrier to reach a target region.

7. The system of claim 1, wherein a diameter of the microcapsule ranges between about 10 um to about 1000 μm.

8. The system of claim 1, wherein the microcapsule is configured to contain the therapeutic agent more than about 73.00% of the microcapsule volume.

9. The system of claim 1, wherein a thickness of the layer ranges from about 10 nm to about 10 μm.

10. The system of claim 1, wherein the therapeutic agent is a water-based therapeutic agent or an oil-based therapeutic agent.

11. The system of claim 1, wherein the motion comprises rolling, walking, kayaking, spinning, or combinations thereof.

12. The system of claim 1, wherein the first type of the nanoparticles is SiO₂ nanoparticle and the second type of nanoparticles is Fe₃O₄ nanoparticle.

13. The system of claim 1, wherein the system is configured to deliver the therapeutic agent without causing surface damages or cytotoxicity.

14. A method of delivering a therapeutic agent comprising:
introducing at least one microcapsule to a subject, wherein the microcapsule comprises the therapeutic agent and a layer of iron oxide nanoparticles (IONPs), wherein the therapeutic agent is encapsulated by the layer of the IONPs, wherein the layer of the IONPs comprises a first type of nanoparticles and a second type of nanoparticles;
directing the at least one microcapsule to a target area using a magnetic system, wherein the magnetic system is configured to generate a magnetic field to control a motion of the at least one microcapsule; and
disrupting the at least one microcapsule for releasing the therapeutic agent at the target area.

15. The method of claim 14, further comprises introducing the therapeutic agent before or after the microcapsule is formed.

16. The method of claim 14, further comprises producing a robotic assembly chain by coupling at least two microcapsules.

17. The method of claim 16, wherein the at least two microcapsules are asymmetric microcapsules.

18. The method of claim 14, wherein the motion comprises rolling, walking, kayaking, spinning, or combinations thereof.

19. The method of claim 14, wherein the directing the at least one microcapsule to the target area comprises delivering the therapeutic agent to the target area without damages or cytotoxicity.

20. The method of claim 14, wherein a structure of the microcapsule is disturbed at a target region by a physical force, a thermal energy, or a magnetic force.

21. The method of claim 18, wherein the directing the at least one microcapsule to the target area comprises directing the at least one microcapsule to overcome physical barriers, fluid shear, confined spaces, sticky tissues, sticky surfaces, or combinations thereof by inducing the motions.

22. The method of claim 14, further comprising accumulating at least two microcapsules to the target area to increase a drug payload release.
